SEQUENCE LISTING (38 sequences)

SEQ ID NO: 1 - DNA, 1011 bp, Candida parapsilosis
atgtcaattccatcaagccagtacggattcgtattcaataagcaatcaggacttaatttgcgcaatgatttgcctgtccacaagcccaaagcgggtcaattgttgttgaaagttgatgctgttggattgtgtcattctgatttacatgtcatttacgaagggttggattgtggtgataattatgtaatgggacatgaaattgctggaactgttgctgctgtgggtgatgatgtcattaactacaaggttggtgatcgtgttgcctgtgtcggacccaatggatgtggtgggtgcaagtattgtcgtggtgccattgacaatgtatgtaaaaacgcatttggtgattggttcggattggggtacgatggtgggtatcaacagtacttgttggttactcgcccacgtaacttgtctcgtatcccagataacgtatctgcagacgtggctgcggcttcaactgatgctgtattgacaccatatcacgcaatcaagatggctcaagtgtcaccaacttcgaatatcttgcttattggtgctggtggattgggtggaaatgcaattcaagttgccaaggcatttggtgcgaaagttactgttttggacaaaaaaaaggaggctcgtgaccaagcaaagaagttgggtgctgatgcagtttatgaaacattgccagaatccatttctcctggctctttttcagcatgttttgattttgtttcagtgcaagctacatttgatgtatgtcaaaagtatgttgaaccaaagggtgtaattatgcccgtgggactcggtgctcctaatttatcgtttaatttgggagatttggcattgcgcgaaattcgaatcttgggtagtttttggggaactactaatgatttggatgatgttttgaaattggttagtgaaggtaaagttaaacccgttgtgcgcagtgccaaattgaaggaattgccagagtatattgaaaaattgcgcaacaatgcttatgaaggtcgtgttgtttttaatccataa

SEQ ID NO: 2 - DNA, 1047 bp, Rhodococcus erythropolis
atgaaagcaatccagtacacgagaatcggcgcggaacccgaactcacggagattcccaagcccgagcccggtccaggtgaagtgctcctggaagtcaccgctgccggcgtctgccactcggacgacttcatcatgagcctgcccgaagagcagtacacctacggccttccgctcacgctcggccacgaaggcgcaggcaaggtcgccgccgtcggcgagggtgtcgaaggtctcgacatcggaaccaatgtcgtcgtctacgggccttggggttgcggcaactgttggcactgctcacaaggactcgagaactattgctctcgcgcccaagaactcggaatcaatcctcccggtctcggtgcacccggcgcgttggccgagttcatgatcgtcgattctcctcgccaccttgtcccgatcggtgacctcgacccggtcaagacggtgccgctgaccgacgccggtctgacgccgtatcacgcgatcaagcgttctctgccgaaacttcgcggaggctcgttcgcggttgtcattggtaccggcggtctcggccacgtcgctattcagctcctccgtcacctctcggcggcaacggtcatcgctttggacgtgagcgcggacaagctcgaactggcaaccaaggtaggcgctcacgaagtggttctgtccgacaaggacgcggccgagaacgtccgcaagatcactggaagtcaaggcgccgcactggttctcgacttcgtcggctaccagcccaccatcgacaccgcgatggctgtcgccggcgtcggatcagacgtcacgatcgtcgggatcggggacggccaggcccacgccaaagtcgggttcttccaaagtccttacgaggcttcggtgacagttccgtattggggtgcccgcaacgagttgatcgaattgatcgacctcgcccacgccggcatcttcgacatcgcggtggagaccttcagtctcgacaacggtgccgaagcgtatcgacgactggctgccggaacgctaagcggccgtgcggttgtggtccctggtctttaa

SEQ ID NO: 3 - DNA, 1041 bp, Streptomyces coelicolor
atgaaagcactgcagtatcgtactattggtgcaccacctgaagttgtaacggtaccagatccagaacctggtccaggtcaagttttacttaaagttactgcggccggtgtgtgccatagcgatatcgctgttatgtcttggcctgccgaaggcttcccgtatgaactgccgctgaccctgggtcacgagggcgtaggtactgtggcagcgctgggcgccggcgttaccggcctggcagaaggcgacgcggtcgctgtgtatggtccgtggggctgtggtacgtgcgccaaatgtgcagaaggcaaagaaaattattgcctgcgtgcggatgagctggggatccgccctccgggtcttggccgtccgggttctatggctgaatacctgctcatcgacgatccgcgccatctcgtgccgctggatggcttagatccggtagcagcggtgccgctgaccgacgctggtttgactccgtaccacgccattaaacgttcactgcctaagctggttccggggtcgaccgcagtcgtgatcgggactggtggccttggtcatgttgcgatccaattgctccgcgctctgacgtcagcgcgtgtagtggcgctggatgtcagcgaagaaaagttacgcctggcacgtgccgtcggcgcacacgaggcggtgttgtctgacgctaaagcagcggatgctgttcgcgaaattaccggtggcctgggggccgaagcagtattcgattttgtgggtgttgcgccaaccgtccagaccgcgggcgcagtggcggctgttgaaggtgacgtaaccctggtgggcatcggtggcgggtccttgccggttggttttggcatgctgccgttcgaagtctcagtgaacgccccgtattggggttcgcgtagtgaactcactgaagttctgaacctggcacgcagcggcgcggtatctgtacataccgaaacgtactccttagatgacgctccgctggcgtacgagcgcttgcacgaaggtcgcgttaatggtcgtgctgttattttaccacatggttaa

SEQ ID NO: 4 - DNA, 780 bp, Zoogloea ramigera
atgtcgttgaatggcaaagtcattttggtaaccggcgctggccaggggatcggacgcggtattgcgctgcggcttgcaaaggaaggcgctgatctcgcgctggccgacgtcaaggccgataagctcgactccgttcgcaaggaagtcgaagcgctcggacgcaaggccaccaccgtcgtcgccgatgtcagcaagcgcgacgaagtctacgccgccatcgaccatgcagagaagcaactcggcgggttcgacgtcatggtcaacaatgccggcatcgcccaggtcaagccgatcgccgacgtcacgcccgaggacatggacctgatcttccggatcaatgtcgacggcgtgctctggggcatccaggcggcttcgcagaaattcaaggatcgaggtcagaagggcaagatcatcaatgcctgctcgattgccggacatgacggcttcgccatgctcggcgtctattcggcaaccaagttcgccgtgcgtgcgctgacgcaagccgccgccaaggaatatgccagcgcgggcatcacggtgaacgcctactgccccggcatcgtcggcaccgacatgtgggtggagatcgacgagcgcttttccgagatcaccggaacgccgaagggcgaaacctacaagaaatacgtcgagggcatcgctttgggccgcgcgcagacaccggaggacgtggcagcgttggtcgccttcctcgcgggcgccgattccgactacatcacgggacagtcgatcctgaccgatggcggtatcgtctatcgataa

SEQ ID NO: 5 - DNA, 939 bp, Saccharomyces cerevisiae
atgcctgctactttacatgattctacgaaaatcctttctctaaatactggagcccaaatccctcaaataggtttaggtacgtggcagtcgaaagagaacgatgcttataaggctgttttaaccgctttgaaagatggctaccgacacattgatactgctgctatttaccgtaatgaagaccaagtcggtcaagccatcaaggattcaggtgttcctcgggaagaaatctttgttactacaaagttatggtgtacacaacaccacgaacctgaagtagcgctggatcaatcactaaagaggttaggattggactacgtagacttatatttgatgcattggcctgccagattagatccagcctacatcaaaaatgaagacatcttgagtgtgccaacaaagaaggatggttctcgtgcagtggatatcaccaattggaatttcatcaaaacctgggaattaatgcaggaactaccaaagactggtaaaactaaggccgttggagtctccaacttttctataaataacctgaaagatctattagcatctcaaggtaataagcttacgccagctgctaaccaagtcgaaatacatccattactacctcaagacgaattgattaatttttgtaaaagtaaaggcattgtggttgaagcttattctccgttaggtagtaccgatgctccactattgaaggaaccggttatccttgaaattgcgaagaaaaataacgttcaacccggacacgttgttattagctggcacgtccaaagaggttatgttgtcttgccaaaatctgtgaatcccgatcgaatcaaaacgaacaggaaaatatttactttgtctactgaggactttgaagctatcaataacatatcgaaggaaaagggcgaaaaaagggttgtacatccaaattggtctcctttcgaagtattcaagtaa

SEQ ID NO: 6 - DNA, 825 bp, Hyoscyamus niger
atggccggagaatcagaagtttacattaatggcaacaatggaggaattagatggagtctcaaaggcacaactgcccttgttactggtggctctaaaggcattgggtatgcagtagtggaagaactagcaggtcttggtgcaagagtatatacatgttcacgtaatgaaaaggaactccaacaatgccttgatatttggagaaatgaaggacttcaagttgaaggttctgtttgtgatttattactgcgctctgaacgtgacaaacttatgcagactgttgcagatttatttaatggaaagctcaatattttggtaaataatgcaggtgtggtgatacataaagaagctaaagatttcacaaaagaagattacgacatcgtattgggcactaattttgaagcagcttatcacttatgtcaacttgcttatccctttttgaaggcatctcaaaatggcaatgttatttttctttcttctatagctggattttcagcactgccttctgtttctctttattctgcttccaaagctgcaataaatcaaataacgaagaacttggcatgtgaatgggccaaggacaacattcgggtcaattcagttgctccaggagtcattttaaccccactcattgaaactgcaattaagaaaaatcctcatcaaaaagaagaaatagacaattttattgtcaagactccaatgggccgggctggaaagcccaatgaagtgtctgcactaatagcctttctttgcttccctgctgcttcttatattactggccaaattatatgggctgatggtggattcacagctaatggtgggttttaa

SEQ ID NO: 7 - DNA, 822 bp, Datura stramonium
atggaagaatcaaaagtgtccatgatgaattgcaacaatgaaggaagatggagtctcaaaggcaccacagcccttgttactggtggctctaaaggcattgggtatgcaatagtggaagaattggcaggtcttggagcaagagtatatacatgttcacgtaatgaaaaagaactggacgaatgccttgaaatttggagagaaaaaggacttaatgttgaaggttctgtttgtgacttattatcacgtactgaacgtgataagcttatgcagactgttgcacatgtatttgatggaaagctcaatattttggtgaataatgccggggtggtgatacataaggaagctaaagatttcacagaaaaagattacaacataattatgggaactaattttgaagcagcttatcatttatctcaaattgcttatccattattgaaggcttctcaaaatgggaatgttatttttctctcttctattgctggattttcagcactgccttctgtttctctttactcagcttccaaaggtgcaataaatcaaatgacaaagagtttggcttgtgaatgggctaaagacaacattcgggtcaattcagttgctccgggagtcattttaaccccactggttgaaactgcaattaagaaaaatcctcatcaaaaagaagaaatagacaattttattgtcaagactcctatgggccgggccggaaagccccaagaagtttctgcactaatagcttttctttgcttccctgctgcttcatatattacgggccagatcatatgggctgacggtggattcacagctaatggtgggttttaa

SEQ ID NO: 8 - DNA, 1014 bp, Geobacillus stearothermophilus
atgaaagctgcagttgtggaacaatttaaaaagccgttacaagtgaaagaagtggaaaaacctaagatctcatacggggaagtattagtgcgcatcaaagcgtgtggggtatgccatacagacttgcatgccgcacatggcgactggcctgtaaagcctaaactgcctctcattcctggccatgaaggcgtcggtgtaattgaagaagtaggtcctggggtaacacatttaaaagttggagatcgcgtaggtatcccttggctttattcggcgtgcggtcattgtgactattgcttaagcggacaagaaacattatgcgaacgtcaacaaaacgctggctattccgtcgatggtggttatgctgaatattgccgtgctgcagccgattatgtcgtaaaaattcctgataacttatcgtttgaagaagccgctccaatcttttgcgctggtgtaacaacatataaagcgctcaaagtaacaggcgcaaaaccaggtgaatgggtagccatttacggtatcggcgggcttggacatgtcgcagtccaatacgcaaaggcgatggggttaaacgtcgttgctgtcgatttaggtgatgaaaaacttgagcttgctaaacaacttggtgcagatcttgtcgtcaatccgaaacatgatgatgcagcacaatggataaaagaaaaagtgggcggtgtgcatgcgactgtcgtcacagctgtttcaaaagccgcgttcgaatcagcctacaaatccattcgtcgcggtggtgcttgcgtactcgtcggattaccgccggaagaaatacctattccaattttcgatacagtattaaatggagtaaaaattattggttctatcgttggtacgcgcaaagacttacaagaggcacttcaatttgcagcagaaggaaaagtaaaaacaattgtcgaagtgcaaccgcttgaaaacattaacgacgtattcgatcgtatgttaaaagggcaaattaacggccgcgtcgtgttaaaagtagattaa

SEQ ID NO: 9 - protein, 336 aa, Candida parapsilosis
MSIPSSQYGFVFNKQSGLNLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 10 - protein, 348 aa, Rhodococcus erythropolis
MKAIQYTRIGAEPELTEIPKPEPGPGEVLLEVTAAGVCHSDDFIMSLPEEQYTYGLPLTLGHEGAGKVAAVGEGVEGLDIGTNVVVYGPWGCGNCWHCSQGLENYCSRAQELGINPPGLGAPGALAEFMIVDSPRHLVPIGDLDPVKTVPLTDAGLTPYHAIKRSLPKLRGGSFAVVIGTGGLGHVAIQLLRHLSAATVIALDVSADKLELATKVGAHEVVLSDKDAAENVRKITGSQGAALVLDFVGYQPTIDTAMAVAGVGSDVTIVGIGDGQAHAKVGFFQSPYEASVTVPYWGARNELIELIDLAHAGIFDIAVETFSLDNGAEAYRRLAAGTLSGRAVVVPGL

SEQ ID NO: 11 - protein, 346 aa, Streptomyces coelicolor
MKALQYRTIGAPPEVVTVPDPEPGPGQVLLKVTAAGVCHSDIAVMSWPAEGFPYELPLTLGHEGVGTVAALGAGVTGLAEGDAVAVYGPWGCGTCAKCAEGKENYCLRADELGIRPPGLGRPGSMAEYLLIDDPRHLVPLDGLDPVAAVPLTDAGLTPYHAIKRSLPKLVPGSTAVVIGTGGLGHVAIQLLRALTSARVVALDVSEEKLRLARAVGAHEAVLSDAKAADAVREITGGLGAEAVFDFVGVAPTVQTAGAVAAVEGDVTLVGIGGGSLPVGFGMLPFEVSVNAPYWGSRSELTEVLNLARSGAVSVHTETYSLDDAPLAYERLHEGRVNGRAVILPHG

SEQ ID NO: 12 - protein, 259 aa, Zoogloea ramigera
MSLNGKVILVTGAGQGIGRGIALRLAKEGADLALADVKADKLDSVRKEVEALGRKATTVVADVSKRDEVYAAIDHAEKQLGGFDVMVNNAGIAQVKPIADVTPEDMDLIFRINVDGVLWGIQAASQKFKDRGQKGKIINACSIAGHDGFAMLGVYSATKFAVRALTQAAAKEYASAGITVNAYCPGIVGTDMWVEIDERFSEITGTPKGETYKKYVEGIALGRAQTPEDVAALVAFLAGADSDYITGQSILTDGGIVYR

SEQ ID NO: 13 - protein, 312 aa, Saccharomyces cerevisiae
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 14 - protein, 274 aa, Hyoscyamus niger
MAGESEVYINGNNGGIRWSLKGTTALVTGGSKGIGYAVVEELAGLGARVYTCSRNEKELQQCLDIWRNEGLQVEGSVCDLLLRSERDKLMQTVADLFNGKLNILVNNAGVVIHKEAKDFTKEDYDIVLGTNFEAAYHLCQLAYPFLKASQNGNVIFLSSIAGFSALPSVSLYSASKAAINQITKNLACEWAKDNIRVNSVAPGVILTPLIETAIKKNPHQKEEIDNFIVKTPMGRAGKPNEVSALIAFLCFPAASYITGQIIWADGGFTANGGF

SEQ ID NO: 15 - protein, 273 aa, Datura stramonium
MEESKVSMMNCNNEGRWSLKGTTALVTGGSKGIGYAIVEELAGLGARVYTCSRNEKELDECLEIWREKGLNVEGSVCDLLSRTERDKLMQTVAHVFDGKLNILVNNAGVVIHKEAKDFTEKDYNIIMGTNFEAAYHLSQIAYPLLKASQNGNVIFLSSIAGFSALPSVSLYSASKGAINQMTKSLACEWAKDNIRVNSVAPGVILTPLVETAIKKNPHQKEEIDNFIVKTPMGRAGKPQEVSALIAFLCFPAASYITGQIIWADGGFTANGGF

SEQ ID NO: 16 - protein, 337 aa, Geobacillus stearothermophilus
MKAAVVEQFKKPLQVKEVEKPKISYGEVLVRIKACGVCHTDLHAAHGDWPVKPKLPLIPGHEGVGVIEEVGPGVTHLKVGDRVGIPWLYSACGHCDYCLSGQETLCERQQNAGYSVDGGYAEYCRAAADYVVKIPDNLSFEEAAPIFCAGVTTYKALKVTGAKPGEWVAIYGIGGLGHVAVQYAKAMGLNVVAVDLGDEKLELAKQLGADLVVNPKHDDAAQWIKEKVGGVHATVVTAVSKAAFESAYKSIRRGGACVLVGLPPEEIPIPIFDTVLNGVKIIGSIVGTRKDLQEALQFAAEGKVKTIVEVQPLENINDVFDRMLKGQINGRVVLKVD

SEQ ID NO: 17 - DNA, 765 bp, Pichia finlandica
atgtcttataacttccataacaaggttgcagttgttactggagctctatcaggaatcggcttaagcgtcgcaaaaaagttccttcagctcggcgccaaagtaacgatctctgatgtcagtggagagaaaaaatatcacgagactgttgttgctctgaaagcccaaaatctcaacactgacaacctccattatgtacaggcagattccagcaaagaagaagataacaagaaattgatttcggaaactctggcaacctttgggggcctggatattgtttgtgctaatgcaggaattggaaagttcgctcccacccatgaaacacccttcgacgtatggaagaaggtgattgctgtgaatttgaatggagtattcttactggataagctagccatcaattactggctagagaaaagcaaacccggcgtaattgtcaacatgggatcagtccactcttttgtagcagctcctggccttgcgcattatggagctgcaaaaggcggtgtcaaactgttaacacaaacattggctctagagtacgcatctcatggtattagagtaaattctgtcaatccggggtacatttcgactcctttgatagatgaggttccgaaagagcggttggataaacttgtaagcttgcaccctattgggagactaggtcgtccagaggaagttgctgatgcagtcgcatttctgtgttcccaggaggccactttcatcaacggcgtttctttgccggttgacggggggtacacagcccagtaa

SEQ ID NO: 18 - protein, 254 aa, Pichia finlandica
MSYNFHNKVAVVTGALSGIGLSVAKKFLQLGAKVTISDVSGEKKYHETVVALKAQNLNTDNLHYVQADSSKEEDNKKLISETLATFGGLDIVCANAGIGKFAPTHETPFDVWKKVIAVNLNGVFLLDKLAINYWLEKSKPGVIVNMGSVHSFVAAPGLAHYGAAKGGVKLLTQTLALEYASHGIRVNSVNPGYISTPLIDEVPKERLDKLVSLHPIGRLGRPEEVADAVAFLCSQEATFINGVSLPVDGGYTAQ

SEQ ID NO: 19 - DNA, 34 bp, Artificial
  An artificially synthesized primer sequence
gtggaattctataatgtcaattccatcaagccag

SEQ ID NO: 20 - DNA, 43 bp, Artificial
  An artificially synthesized primer sequence
ctgaagcttattatggattaaaaacaacacgaccttcataagc

SEQ ID NO: 21 - DNA, 34 bp, Artificial
  An artificially synthesized primer sequence
cacgaattctatcatgaaagcaatccagtacacg

SEQ ID NO: 22 - DNA, 35 bp, Artificial
  An artificially synthesized primer sequence
tcgaagcttctagattaaagaccagggaccacaac

SEQ ID NO: 23 - DNA, 37 bp, Artificial
  An artificially synthesized primer sequence
gagccatggcacctgctactttacatgattctacgaa

SEQ ID NO: 24 - DNA, 42 bp, Artificial
  An artificially synthesized primer sequence
gagcttaagtctagattatttgaatacttcgaaaggagacca

SEQ ID NO: 25 - DNA, 31 bp, Artificial
  An artificially synthesized primer sequence
gaggaattcaatcatgaaagctgcagttgtg

SEQ ID NO: 26 - DNA, 37 bp, Artificial
  An artificially synthesized primer sequence
gtcaagcttctagattaatctacttttaacacgacgc

SEQ ID NO: 27 - DNA, 39 bp, Artificial
  An artificially synthesized primer sequence
gtcgaattcatacatgtctatcccagaaactcaaaaagg

SEQ ID NO: 28 - DNA, 47 bp, Artificial
  An artificially synthesized primer sequence
ctgcttaagtctagattatttagaagtgtcaacaacgtaacgaccaa

SEQ ID NO: 29 - DNA, 1047 bp, Saccharomyces cerevisiae
atgtctatcccagaaactcaaaaaggtgttatcttctacgaatcccacggtaaattggaacacaaggatattccagttccaaagccaaaggccaacgaattgttgatcaacgttaagtactctggtgtctgtcacaccgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcattgggttgccatttccggtgctgccggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagccgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 30 - protein, 348 aa, Saccharomyces cerevisiae
MSIPETQKGVIFYESHGKLEHKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 31 - DNA, 39 bp, Artificial
  An artificially synthesized primer sequence
gtcgaattcatacatgtctattccagaaactcaaaaagc

SEQ ID NO: 32 - DNA, 47 bp, Artificial
  An artificially synthesized primer sequence
gcacttaagtctagattatttagaagtgtcaacaacgtaacgaccag

SEQ ID NO: 33 - DNA, 1047 bp, Saccharomyces cerevisiae
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtcgttacgttgttgacacttctaaataa

SEQ ID NO: 34 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 35 - DNA, 36 bp, Artificial
  An artificially synthesized primer sequence
gagtcatgagttcactggttactcttaataacggtc

SEQ ID NO: 36 - DNA, 43 bp, Artificial
  An artificially synthesized primer sequence
gacgaattcctctagattatgcaaaagtggggaatttaccatc

SEQ ID NO: 37 - DNA, 984 bp, Saccharomyces cerevisiae
atgagttcactggttactcttaataacggtctgaaaatgcccctagtcggcttagggtgctggaaaattgacaaaaaagtctgtgcgaatcaaatttatgaagctatcaaattaggctaccgtttattcgatggtgcttgcgactacggcaacgaaaaggaagttggtgaaggtatcaggaaagccatctccgaaggtcttgtttctagaaaggatatatttgttgtttcaaagttatggaacaattttcaccatcctgatcatgtaaaattagctttaaagaagaccttaagcgatatgggacttgattatttagacctgtattatattcacttcccaatcgccttcaaatatgttccatttgaagagaaataccctccaggattctatacgggcgcagatgacgagaagaaaggtcacatcaccgaagcacatgtaccaatcatagatacgtaccgggctctggaagaatgtgttgatgaaggcttgattaagtctattggtgtttccaactttcagggaagcttgattcaagatttattacgtggttgtagaatcaagcccgtggctttgcaaattgaacaccatccttatttgactcaagaacacctagttgagttttgtaaattacacgatatccaagtagttgcttactcctccttcggtcctcaatcattcattgagatggacttacagttggcaaaaaccacgccaactctgttcgagaatgatgtaatcaagaaggtctcacaaaaccatccaggcagtaccacttcccaagtattgcttagatgggcaactcagagaggcattgccgtcattccaaaatcttccaagaaggaaaggttacttggcaacctagaaatcgaaaaaaagttcactttaacggagcaagaattgaaggatatttctgcactaaatgccaacatcagatttaatgatccatggacctggttggatggtaaattccccacttttgcataa

SEQ ID NO: 38 - protein, 327 aa, Saccharomyces cerevisiae
MSSLVTLNNGLKMPLVGLGCWKIDKKVCANQIYEAIKLGYRLFDGACDYGNEKEVGEGIRKAISEGLVSRKDIFVVSKLWNNFHHPDHVKLALKKTLSDMGLDYLDLYYIHFPIAFKYVPFEEKYPPGFYTGADDEKKGHITEAHVPIIDTYRALEECVDEGLIKSIGVSNFQGSLIQDLLRGCRIKPVALQIEHHPYLTQEHLVEFCKLHDIQVVAYSSFGPQSFIEMDLQLAKTTPTLFENDVIKKVSQNHPGSTTSQVLLRWATQRGIAVIPKSSKKERLLGNLEIEKKFTLTEQELKDISALNANIRFNDPWTWLDGKFPTFA

(19) United States
(12) Patent Application Publication
(10) Pub. No.: US 2009/0203096 A1
(43) Pub. Date: Aug. 13, 2009

(54) Process for Production of Optically Active Alcohol

(75) Inventors: Motoko Hayashi, Hyogo (JP); Teruyuki Nikaido, Hyogo (JP); Hiroaki Yamamoto, Hyogo (JP)

Correspondence Address: FOLEY AND LARDNER LLP;SUITE 500, 3000 K STREET NW, WASHINGTON, DC, 20007, US

(73) Assignee: DAICEL CHEMICAL INDUSTRIES, LTD., Osaka (JP)

(21) Appl. No.: 12/227,981

(22) PCT Filed: Jun. 5, 2007
(86) PCT No.: PCT/JP2007/061325

§ 371 Date: Apr. 6, 2009

(30) Foreign Application Priority Data
Jun. 5, 2006 (JP) 2006-156059

(51) Int. Cl. C12P 7/04

(52) U.S. Cl. 435/157

(57) ABSTRACT

The present invention provides methods for producing (S)-1,1,1-trifluoro-2-propanol, which include the step of reacting an enzyme of any one of alcohol dehydrogenase CpSADH, alcohol dehydrogenase ReSADH, carbonyl reductase ScoPAR, (2S,3S)-butanediol dehydrogenase ZraSBDH, carbonyl reductase ScGCY1, tropinone reductase HnTR1, tropinone reductase DsTR1, or alcohol dehydrogenase BstADHT, a microorganism or a transformant strain that functionally expresses the enzyme, or a processed material thereof, with 1,1,1-trifluoroacetone. The present invention also provides methods for producing (R)-1,1,1-trifluoro-2-propanol, which include the step of reacting alcohol dehydrogenase PfODH, a microorganism or a transformant strain that functionally expresses the enzyme, or a processed material thereof, with 1,1,1-trifluoroacetone.

### TECHNICAL FIELD

The present invention relates to methods for producing optically active fluorine-containing compounds that are useful as optically active raw materials for various types of pharmaceutical products, liquid crystalline materials, and such.

### BACKGROUND ART

The methods described below in <1> to <4> are known as methods for producing (S)-1,1,1-trifluoro-2-propanol represented by formula (2)

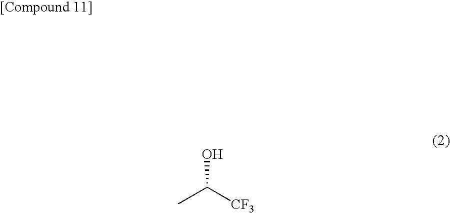

and (R)-1,1,1-trifluoro-2-propanol represented by formula (3).

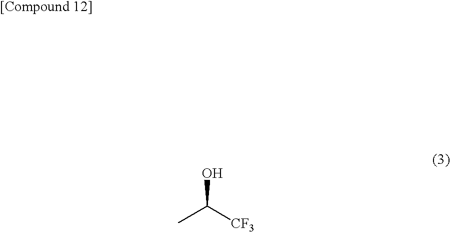

<1> the method for asymmetrically reducing 1,1,1-trifluoroacetone represented by formula (1)

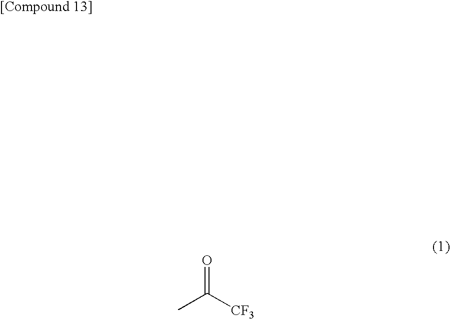

using baker's yeast (Non-Patent Document 1);
<2> the method for asymmetrically reducing 1,1,1-trifluoroacetone represented by formula (1) using DIP-C1, which is a chiral borane reducing agent (Non-Patent Document 2);
<3> the method for obtaining an optically active alcohol by asymmetrically reducing 1,1,1-trifluoro-3-bromoacetone using DIP-C1, which is a chiral borane reducing agent, then using a base to generate an optically active epoxide, which is then subjected to ring-opening using a hydride (Non-Patent Document 3); and
<4> the method for obtaining an optically active alcohol by carrying out a kinetic optical resolution by performing a hydrolysis reaction using lipase on esters of a racemic mixture of the alcohols represented by formulas (2) and (3) (Non-Patent Document 4).

However, the methods of <1>, <2>, and <4> all have low optical purity. In the method of <1>, the substrate concentration is very low being 0.3%, but yet uses baker's yeast as a catalyst at 18% in water, which is the solvent. It also uses the auxiliary material, glucose, at 21% which is a large amount compared to the substrate. Thus, the method of <1> has very poor efficiency. The methods of <2> and <3> require expensive reducing agents. The method of <3> involves complicated steps. The method of <4> gives a theoretical yield less than 50%. Therefore, all of the methods carry issues as industrial production methods.

Information on prior art documents relating to the invention of this application is shown below.

### [Non-Patent Document 1] Synthesis, 897-899 (1983).

### [Non-Patent Document 2] Tetrahedron, 49 (9), 1725-1738 (1993).

[Non-Patent Document 3] J. Org. Chem., 60 (1), 41-46 (1995).
[Non-Patent Document 4] Chem. Lett., 855-856 (1996).

### DISCLOSURE OF THE INVENTION

### Problems to be Solved by the Invention

The present invention was achieved in view of the above-mentioned problems. An objective of the present invention is to provide novel methods for producing optically active alcohols represented by formulas (2) and (3). More specifically, an objective is to provide methods for producing optically active alcohols represented by formulas (2) and (3) at a good yield, as well as high purity.

### Means for Solving the Problems

The present inventors examined methods for producing optically active alcohols with high optical purity, not by a resolution technique which gives a theoretical yield of less than 50%, but by reducing a ketone, where 100% of the raw material can be used, by an asymmetric reduction reaction using a highly stereoselective enzyme.

The present inventors used various types of enzymes that asymmetrically reduce a carbonyl with 1,1,1-trifluoroacetone (hereinafter abbreviated as TFAC) represented by formula (1) as the substrate, and examined methods for synthesizing an optically active 1,1,1-trifluoro-2-propanol (hereinafter abbreviated as TFIP) represented by formula (2) or (3). As a result, the present inventors succeeded in discovering that every one of the following produces (S)-TFIP with very high stereoselectivity of 90% e.e. or more:

alcohol dehydrogenase CpSADH (a protein comprising the amino acid sequence of SEQ ID NO: 9) derived from Candida parapsilosis;
alcohol dehydrogenase ReSADH (a protein comprising the amino acid sequence of SEQ ID NO: 10) derived from Rhodococcus erythropolis;
carbonyl reductase ScoPAR (a protein comprising the amino acid sequence of SEQ ID NO: 11) derived from Streptomyces coelicolor;
(2S,3S)-butanediol dehydrogenase ZraSBDH (a protein comprising the amino acid sequence of SEQ ID NO: 12) derived from Zoogloea ramigera;
carbonyl reductase ScGCY1 (a protein comprising the amino acid sequence of SEQ ID NO: 13) derived from Saccharomyces cerevisiae;
tropinone reductase HnTR1 (a protein comprising the amino acid sequence of SEQ ID NO: 14) derived from Hyoscyamus niger;
tropinone reductase DsTR1 (a protein comprising the amino acid sequence of SEQ ID NO: 15) derived from Datura stramonium; and
alcohol dehydrogenase BstADHT (a protein comprising the amino acid sequence of SEQ ID NO: 16) derived from Geobacillus stearothermophilus. Furthermore, the present inventors succeeded in discovering that alcohol dehydrogenase PfODH (a protein comprising the amino acid sequence of SEQ ID NO: 18) derived from Pichia finlandica produces (R)-TFIP with very high stereoselectivity of 95% e.e. or more.

It had been known that CpSADH (Japanese Patent No. 3574682) produces (S)-1,3-butanediol by asymmetric reduction of 4-hydroxy-2-butanone, ReSADH (Appl. Microbiol. Biotechnol., 62, 380-386 (2003)) and ScoPAR (Japanese Patent Application Kokai Publication No. (JP-A) 2005-95022 (unexamined, published Japanese patent application)) produces (S)-2-octanol by asymmetric reduction of 2-octanone, ZraSBDH (JP-A (Kokai) 2004-357639) produces (2S,3S)-butanediol by asymmetric reduction of 2,3-butanedione, ScGCY1 (FEBS Lett., 238, 123-128, (1988)) produces ethyl(S)-hydroxybutanoate by asymmetric reduction of ethyl acetoacetate, HnTR1 and DsTR1 (both described in JP-A (Kokai) 2003-230398) produce tropine by asymmetric reduction of tropinone, BstADHT (FEBS Lett., 33, 1-3, (1973)) produces ethanol by reducing acetaldehyde, and PfODH (WO 01-061014) produces (R)-2-octanol by asymmetric reduction of 2-octanone. However, whether these enzymes have activity towards TFAC, and if they do have activity, with what degree of stereoselectivity they will reduce TFAC, and whether they will produce the (S) or (R)-type configuration were impossible to predict. Therefore, discovering that (S)-TFIP or (R)-TFIP is produced with high selectivity of 90% e.e. or more was a surprising result.

When various enzymes that act on ketones, for example, alcohol dehydrogenase ScADH1 and ScADH2 derived from Saccharomyces cerevisiae (Arch. Biochem. Biophys., 126, 933-944 (1968)) and carbonyl reductase ScGRE3 derived from Saccharomyces cerevisiae (J. Org. Chem., 63, 4996-5000, (1998)) were made to exhibit their effects, the optical purities of the obtained (S)-TFIP were 82.3% e.e., 58.0% e.e., and 69.6% e.e., respectively.

The present invention was achieved in view of the above circumstances, and provides the following [1] to [6]:

[1] a method for producing (S)-1,1,1-trifluoro-2-propanol represented by formula (2),

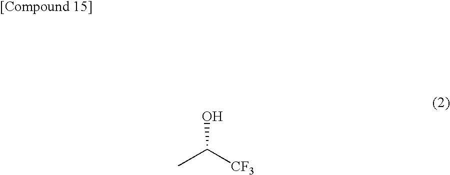

which comprises the step of reacting a protein encoded by the polynucleotide of any one of the following (a) to (e), a microorganism or a transformant strain that functionally expresses said protein, or a processed material thereof, with 1,1,1-trifluoroacetone represented by formula (1):

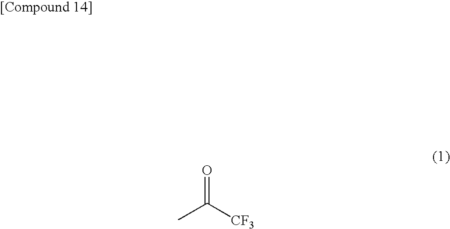

(a) a polynucleotide comprising the nucleotide sequence of any one of SEQ ID NOs: 1 to 8;
(b) a polynucleotide encoding a protein comprising the amino acid sequence of any one of SEQ ID NOs: 9 to 16;
(c) a polynucleotide encoding a protein comprising amino acids with one or more amino acid substitutions, deletions, insertions, and/or additions in the amino acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2);
(d) a polynucleotide that hybridizes under stringent conditions with a DNA comprising the nucleotide sequence of any one of SEQ ID NOs: 1 to 8, wherein the polynucleotide encodes a protein having an activity of reducing 1,1,1-trifluoroacetone represented by formula (1) to produce (S)-1,1,1-trifluoro-2-propanol represented by formula (2); and
(e) a polynucleotide encoding a protein comprising an amino acid sequence having 80% or more homology to the amino-acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2);
[2] a method for producing (R)-1,1,1-trifluoro-2-propanol represented by formula (3),

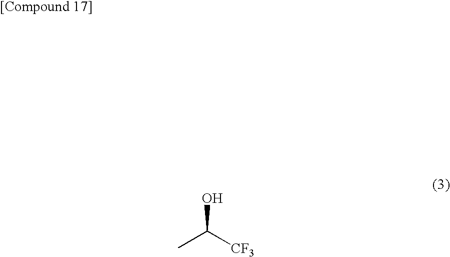

which comprises the step of reacting a protein encoded by the polynucleotide of any one of the following (a) to (e), a microorganism or a transformant strain that functionally expresses said protein, or a processed material thereof, with 1,1,1-trifluoroacetone represented by formula (1):

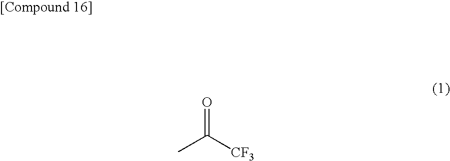

(a) a polynucleotide comprising the nucleotide sequence of SEQ ID NO: 17;
(b) a polynucleotide encoding a protein comprising the amino acid sequence of SEQ ID NO: 18;
(c) a polynucleotide encoding a protein comprising amino acids with one or more amino acid substitutions, deletions, insertions, and/or additions in the amino acid sequence of SEQ ID NO: 18, wherein the protein has an activity of reducing 1,1,1-trifluoroacetone represented by formula (1) to produce (R)-1,1,1-trifluoro-2-propanol represented by formula (3);
(d) a polynucleotide that hybridizes under stringent conditions with a DNA comprising the nucleotide sequence of SEQ ID NO: 17, wherein the polynucleotide encodes a protein having an activity of reducing 1,1,1-trifluoroacetone represented by formula (1) to produce (R)-1,1,1-trifluoro-2-propanol represented by formula (3); and
(e) a polynucleotide encoding a protein comprising an amino acid sequence having 80% or more homology to the amino acid sequence of SEQ ID NO: 18, wherein the protein has an activity of reducing 1,1,1-trifluoroacetone represented by formula (1) to produce (R)-1,1,1-trifluoro-2-propanol represented by formula (3);
[3] a method for producing (S)-1,1,1-trifluoro-2-propanol represented by formula (2),

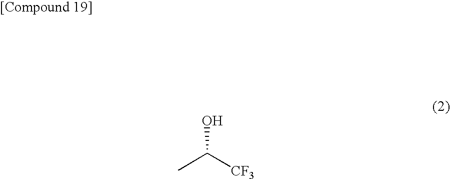

which comprises the step of reacting a protein encoded by the polynucleotide of any one of the following (a) to (e), a transformant strain that coexpresses a coenzyme corresponding to said protein and a dehydrogenase having an activity to regenerate reduced nicotinamide adenine dinucleotide (NADH) or reduced nicotinamide adenine dinucleotide phosphate (NADPH), or a processed material thereof, with 1,1,1-trifluoroacetone represented by formula (1):

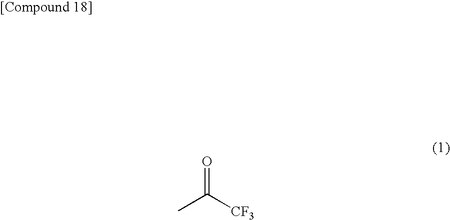

(a) a polynucleotide comprising the nucleotide sequence of any one of SEQ ID NOs: 1 to 8;
(b) a polynucleotide encoding a protein comprising the amino acid sequence of any one of SEQ ID NOs: 9 to 16;
(c) a polynucleotide encoding a protein comprising amino acids with one or more amino acid substitutions, deletions, insertions, and/or additions in the amino acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2);
(d) a polynucleotide that hybridizes under stringent conditions with a DNA comprising the nucleotide sequence of any one of SEQ ID NOs: 1 to 8, wherein the polynucleotide encodes a protein having an activity of reducing 1,1,1-trifluoroacetone represented by formula (1) to produce (S)-1,1,1-trifluoro-2-propanol represented by formula (2); and
(e) a polynucleotide encoding a protein comprising an amino acid sequence having 80% or more homology to the amino acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2);
[4] a method for producing (R)-1,1,1-trifluoro-2-propanol represented by formula (3),

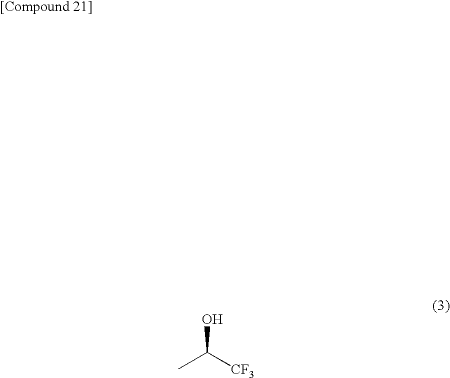

which comprises the step of reacting a protein encoded by the polynucleotide of any one of the following (a) to (e), a transformant strain that coexpresses a coenzyme corresponding to said protein and a dehydrogenase having an activity to regenerate reduced nicotinamide adenine dinucleotide (NADH) or reduced nicotinamide adenine dinucleotide phosphate (NADPH), or a processed material thereof, with 1,1,1-trifluoroacetone represented by formula (1):

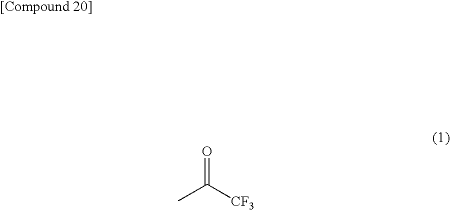

(a) a polynucleotide comprising the nucleotide sequence of SEQ ID NO: 17;
(b) a polynucleotide encoding a protein comprising the amino acid sequence of SEQ ID NO: 18;
(c) a polynucleotide encoding a protein comprising amino acids with one or more amino acid substitutions, deletions, insertions, and/or additions in the amino acid sequence of SEQ ID NO: 18, wherein the protein has an activity of reducing 1,1,1-trifluoroacetone represented by formula (1) to produce (R)-1,1,1-trifluoro-2-propanol represented by formula (3);
(d) a polynucleotide that hybridizes under stringent conditions with a DNA comprising the nucleotide sequence of SEQ ID NO: 17, wherein the polynucleotide encodes a protein having an activity of reducing 1,1,1-trifluoroacetone represented by formula (1) to produce (R)-1,1,1-trifluoro-2-propanol represented by formula (3); and
(e) a polynucleotide encoding a protein comprising an amino acid sequence having 80% or more homology to the amino acid sequence of SEQ ID NO: 18, wherein the protein has an activity of reducing 1,1,1-trifluoroacetone represented by formula (1) to produce (R)-1,1,1-trifluoro-2-propanol represented by formula (3);
[5] the method of [3] or [4], wherein the dehydrogenase having an activity to regenerate reduced nicotinamide adenine dinucleotide (NADH) or reduced nicotinamide adenine dinucleotide phosphate (NADPH) is glucose dehydrogenase or formate dehydrogenase; and
[6] a method for stably producing (S)-1,1,1-trifluoro-2-propanol represented by formula (2)

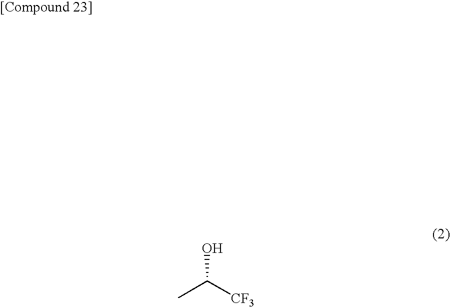

with an optical purity of 99.5% e.e. or more by reacting a protein encoded by the polynucleotide of any one of the following (a) to (e), a microorganism or a transformant strain that functionally expresses said protein, or a processed material thereof, with 1,1,1-trifluoroacetone represented by formula (1) within a pH range of 5.0 to 6.4:

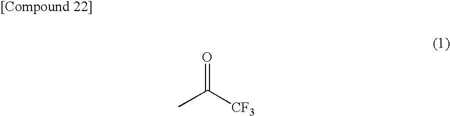

(a) a polynucleotide comprising the nucleotide sequence of SEQ ID NO: 1;
(b) a polynucleotide encoding a protein comprising the amino acid sequence of SEQ ID NO: 9;
(c) a polynucleotide encoding a protein comprising amino acids with one or more amino acid substitutions, deletions, insertions, and/or additions in the amino acid sequence of SEQ ID NO: 9, wherein the protein has an activity of reducing 1,1,1-trifluoroacetone represented by formula (1) to produce (S)-1,1,1-trifluoro-2-propanol represented by formula (2);
(d) a polynucleotide that hybridizes under stringent conditions with a DNA comprising the nucleotide sequence of SEQ ID NO: 1, wherein the polynucleotide encodes a protein having an activity of reducing 1,1,1-trifluoroacetone represented by formula (1) to produce (S)-1,1,1-trifluoro-2-propanol represented by formula (2); and
(e) a polynucleotide encoding a protein comprising an amino acid sequence having 80% or more homology to the amino acid sequence of SEQ ID NO: 9, wherein the protein has an activity of reducing 1,1,1-trifluoroacetone represented by formula (1) to produce (S)-1,1,1-trifluoro-2-propanol represented by formula (2).

### BRIEF DESCRIPTION OF THE DRAWINGS

FIG. 1 depicts the restriction enzyme map of plasmid pSF-CPA4 constructed in Example 1. In the figure, CpSADH refers to the Candida parapsilosis-derived alcohol dehydrogenase gene, McFDH refers to the Mycobacterium vaccae-derived formate dehydrogenase gene, lacI^{q} refers to lac repressor, P(trc) refers to trc promoter, mc refers to multicloning site, T(rrnB) refers to rrnB terminator, amp refers to ampicillin resistance gene, ori refers to replication origin, and rop refers to rop protein gene.

FIG. 2 depicts the restriction enzyme map of plasmid pSE-RED1 constructed in Example 4. ReSADH refers to the Rhodococcus erythropolis-derived alcohol dehydrogenase gene.

FIG. 3 depicts the restriction enzyme map of plasmid pSF-RED1 constructed in Example 5.

FIG. 4 depicts the restriction enzyme map of plasmid pSE-SCP7 constructed in Example 6. ScoPAR refers to the Streptomyces coelicolor-derived carbonyl reductase gene.

FIG. 5 depicts the restriction enzyme map of plasmid pSF-SCP7 constructed in Example 8.

FIG. 6 depicts the restriction enzyme map of plasmid pSE-GCY1 constructed in Example 11. ScGCY refers to the Saccharomyces cerevisiae-derived carbonyl reductase gene.

FIG. 7 depicts the restriction enzyme map of plasmid pSG-GCY1 constructed in Example 12. BsGDH refers to the Bacillus subtilis-derived glucose dehydrogenase gene.

FIG. 8 depicts the restriction enzyme map of plasmid pSE-BSA1 constructed in Example 14. BstADHT refers to the Geobacillus thermocatenulas-derived alcohol dehydrogenase gene.

FIG. 9 depicts the restriction enzyme map of plasmid pSU-SCA1 constructed in Example 16. ScADH1 refers to the Saccharomyces cerevisiae-derived alcohol dehydrogenase I gene.

FIG. 10 depicts the restriction enzyme map of plasmid pSU-SCA2 constructed in Example 18. ScADH2 refers to the Saccharomyces cerevisiae-derived alcohol dehydrogenase II gene.

FIG. 11 depicts the restriction enzyme map of plasmid pSE-GRE3 constructed in Example 20. ScGRE3 refers to the Saccharomyces cerevisiae-derived carbonyl reductase gene.

### MODE FOR CARRYING OUT THE INVENTION

The present invention relates to methods for enzymatically producing optically active (S)-1,1,1-trifluoro-2-propanol. The present invention is based on the discovery made by the present inventors that (S)-1,1,1-trifluoro-2-propanol represented by formula (2)

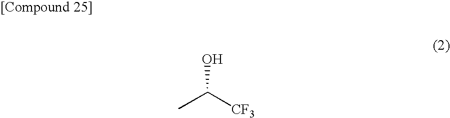

can be produced efficiently by reacting an enzyme of the present invention, a microorganism or a transformant strain that functionally expresses the enzyme, or a processed substance thereof, with 1,1,1-trifluoroacetone represented by formula (1).

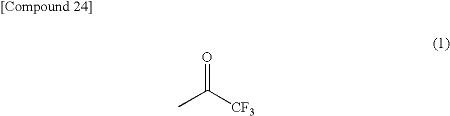

Enzymes that catalyze the above-mentioned reaction from formula (1) to formula (2) include:

alcohol dehydrogenase CpSADH,

alcohol dehydrogenase ReSADH,

carbonyl reductase ScoPAR,

(2S,3S)-butanediol dehydrogenase ZraSBDH,

carbonyl reductase ScGCY1,

tropinone reductase HnTR1,

tropinone reductase DsTR1, and

alcohol dehydrogenase BstADHT.

The present invention further relates to methods for enzymatically producing optically active (R)-1,1,1-trifluoroacetone. This invention is based on the discovery made by the present inventors that (R)-1,1,1-trifluoro-2-propanol represented by formula (3)

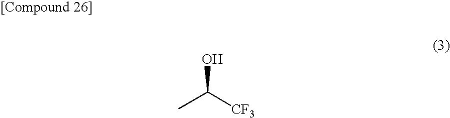

can be produced efficiently by reacting an enzyme of the present invention, a microorganism or a transformant strain that functionally expresses the enzyme, or a processed substance thereof, with 1,1,1-trifluoroacetone represented by formula (1).

Herein, enzymes that catalyze the above-mentioned reaction from formula (1) to formula (3) include alcohol dehydrogenase PfODH. In the present specification, enzymes that catalyze the reaction from formula (1) to formula (2), and enzymes that catalyze the reaction from formula (1) to formula (3) are referred to as “enzymes having carbonyl reductase activity”.

### Enzymes

Hereafter, enzymes of the present invention (alcohol dehydrogenase CpSADH, alcohol dehydrogenase ReSADH, carbonyl reductase ScoPAR, (2S,3S)-butanediol dehydrogenase ZraSBDH, carbonyl reductase ScGCY1, tropinone reductase HnTR1, tropinone reductase DsTR1, alcohol dehydrogenase BstADHT, and alcohol dehydrogenase PfODH) will be described.

First, alcohol dehydrogenase CpSADH in the present invention includes proteins comprising the amino acid sequence of SEQ ID NO: 9. A nucleotide sequences encoding this amino acid sequence include the nucleotide sequence of SEQ ID NO: 1. Alcohol dehydrogenase CpSADH of the present invention includes proteins encoded by:

(1) a polynucleotide encoding a protein comprising amino acids with one or more amino acid substitutions, deletions, insertions, and/or additions in the amino acid sequence of SEQ ID NO: 9, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 1, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 9, wherein the protein has an activity of reducing 1,1,1-trifluoroacetone represented by formula (1) to produce (S)-1,1,1-trifluoro-2-propanol represented by formula (2).

Carbonyl reductase CpSADH used in the present invention can be prepared from Candida parapsilosis by the method described in Japanese Patent No. 3574682. Alternatively, the enzyme can be obtained from a recombinant by cloning, from Candida parapsilosis or such by PCR, the DNA of SEQ ID NO: 1 which encodes the Candida parapsilosis-derived CpSADH, and then expressing CpSADH using a recombinant prepared by introducing the DNA in an expressible form to a heterologous host, such as E. coli.

The enzyme activity of CpSADH in the present invention can be measured, for example, as described below but it is not limited thereto. More specifically, an example includes a method in which a reaction is carried out at 30° C. in a reaction solution containing 100 mM Tris-HCl buffer (pH 9.0), 2.5 mM NAD⁺, 50 mM (S)-1,3-butanediol, and the enzyme and the increase in absorbance at 340 nm, resulting from the production of NADH, is measured. In this case, 1 U is defined as the amount of enzyme that catalyzes the production of 1 μmol of NADH in one minute.

Next, alcohol dehydrogenase ReSADH will be described. Alcohol dehydrogenase ReSADH in the present invention includes proteins comprising the amino acid sequence of SEQ ID NO: 10, and nucleotide sequences encoding this amino acid sequence include the nucleotide sequence of SEQ ID NO: 2. Alcohol dehydrogenase ReSADH of the present invention includes proteins encoded by:

(1) a polynucleotide encoding a protein comprising amino acids with one or more amino acid substitutions, deletions, insertions, and/or additions in the amino acid sequence of SEQ ID NO: 10, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 2, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 10, wherein the protein has an activity of reducing 1,1,1-trifluoroacetone represented by formula (1) to produce (S)-1,1,1-trifluoro-2-propanol represented by formula (2).

Alcohol dehydrogenase ReSADH used in the present invention can be prepared from Rhodococcus erythropolis by the method described in Appl. Microbiol. Biotechnol., 62, 380-386 (2003). Alternatively, the enzyme can be obtained from a recombinant by cloning from Rhodococcus erythropolis or such by PCR the DNA of SEQ ID NO: 2 which encodes the Rhodococcus erythropolis-derived ReSADH, and then expressing ReSADH using a recombinant prepared by introducing the DNA in an expressible form to a heterologous host, such as E. coli.

The enzyme activity of ReSADH in the present invention can be measured, for example, as described below but it is not limited thereto. More specifically, a reaction is carried out at 30° C. in a reaction solution containing 100 mM Tris-HCl buffer (pH 9.0), 2.5 mM NAD⁺, 5 mM (S)-2-octanol, and the enzyme and the increase in absorbance at 340 nm, resulting from the production of NADH, is measured. In this case, 1 U is defined as the amount of enzyme that catalyzes the production of 1 μmol of NADH in one minute.

Next, carbonyl reductase ScoPAR will be described. Carbonyl reductase ScoPAR in the present invention includes proteins comprising the amino acid sequence of SEQ ID NO: 11. Nucleotide sequences encoding this amino acid sequence include the nucleotide sequence of SEQ ID NO: 3. Carbonyl reductase ScoPAR of the present invention includes proteins encoded by:

(1) a polynucleotide encoding a protein comprising amino acids with one or more amino acid substitutions, deletions, insertions, and/or additions in the amino acid sequence of SEQ ID NO: 11, wherein the protein has an activity of reducing 1,1,1-trifluoroacetone represented by formula (1) to produce (S)-1,1-trifluoro-2-propanol represented by formula (2);
(2) a polynucleotide that hybridizes under stringent conditions with a DNA comprising the nucleotide sequence of SEQ ID NO: 3, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 11, wherein the protein has an activity of reducing 1,1,1-trifluoroacetone represented by formula (1) to produce (S)-1,1,1-trifluoro-2-propanol represented by formula (2).

Carbonyl reductase ScoPAR used in the present invention can be prepared from Streptomyces coelicolor by the method described in JP-A (Kokai) 2005-95022. Alternatively, the enzyme can be obtained from a recombinant by cloning from Streptomyces coelicolor or such by PCR the DNA of SEQ ID NO: 3 which encodes the Streptomyces coelicolor-derived ScoPAR, and then expressing ScoPAR using a recombinant prepared by introducing the DNA in an expressible form to a heterologous host, such as E. coli.

The enzyme activity of ScoPAR in the present invention can be measured, for example, as described below but it is not limited thereto. More specifically, a reaction is carried out at 30° C. in a reaction solution containing 100 mM Tris-HCl buffer (pH 9.0), 2.5 mM NAD⁺, 5 mM (S)-2-octanol, and the enzyme and the increase in absorbance at 340 mm, resulting from the production of NADH, is measured. 1 U is defined as the amount of enzyme that catalyzes the production of 1 μmol of NADH in one minute.

Next, (2S,3S)-butanediol dehydrogenase ZraSBDH will be described. (2S,3S)-butanediol dehydrogenase ZraSBDH in the present invention includes proteins comprising the amino acid sequence of SEQ ID NO: 12, and nucleotide sequences encoding this amino acid sequence include the nucleotide sequence of SEQ ID NO: 4. (2S,3S)-butanediol dehydrogenase ZraSBDH of the present invention includes proteins encoded by the following (1) to (3):

(1) a polynucleotide encoding a protein comprising amino acids with one or more amino acid substitutions, deletions, insertions, and/or additions in the amino acid sequence of SEQ ID NO: 12, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 4, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 12, wherein the protein has an activity of reducing 1,1,1-trifluoroacetone represented by formula (1) to produce (S)-1,1,1-trifluoro-2-propanol represented by formula (2).

(2S,3S)-butanediol dehydrogenase ZraSBDH used in the present invention can be prepared from Zoogloea ramigera by the method described in JP-A (Kokai) 2004-357639. Alternatively, the enzyme can be obtained from a recombinant by cloning from Zoogloea ramigera or such by PCR the DNA of SEQ ID NO: 4 which encodes the Zoogloea ramigera-derived ZraSBDH, and then expressing ZraSBDH using a recombinant prepared by introducing the DNA in an expressible form to a heterologous host, such as E. coli.

The enzyme activity of ZraSBDH in the present invention can be measured, for example, as described below but it is not limited thereto. More specifically, a reaction is carried out at 30° C. in a reaction solution containing 100 mM phosphate buffer (pH 8.0), 2.5 mM NAD⁺, 50 mM (2S,3S)-butanediol, and the enzyme, and the increase in absorbance at 340 nm, resulting from the production of NADH, is measured. 1 U is defined as the amount of enzyme that catalyzes the production of 1 μmol of NADH in one minute.

Next, carbonyl reductase ScGCY1 will be described. Carbonyl reductase ScGCY1 in the present invention includes proteins comprising the amino acid sequence of SEQ ID NO: 13. Nucleotide sequences encoding this amino acid sequence include the nucleotide sequence of SEQ ID NO: 5. Carbonyl reductase ScGCY1 of the present invention includes proteins encoded by:

(1) a polynucleotide encoding a protein comprising amino acids with one or more amino acid substitutions, deletions, insertions, and/or additions in the amino acid sequence of SEQ ID NO: 13, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 5, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 13, wherein the protein has an activity of reducing 1,1,1-trifluoroacetone represented by formula (1) to produce (S)-1,1,1-trifluoro-2-propanol represented by formula (2).

Carbonyl reductase ScGCY1 used in the present invention can be obtained from a recombinant by cloning from Saccharomyces cerevisiae or such by PCR the DNA of SEQ ID NO: 5 which encodes the Saccharomyces cerevisiae-derived ScGCY1, and then expressing ScGCY1 using a recombinant prepared by introducing the DNA in an expressible form to a heterologous host, such as E. coli.

The enzyme activity of ScGCY1 in the present invention can be measured, for example, as described below but it is not limited thereto. More specifically, a reaction is carried out at 30° C. in a reaction solution containing 100 mM phosphate buffer (pH 6.5), 0.2 mM NADPH, 20 mM ethyl acetoacetate, and the enzyme, and the decrease in absorbance at 340 nm, resulting from the decrease of NADPH, is measured. 1 U is defined as the amount of enzyme that catalyzes the decrease of 1 μmol of NADH in one minute.

Next, tropinone reductase HnTR1 will be described. Tropinone reductase HnTR1 in the present invention includes proteins comprising the amino acid sequence of SEQ ID NO: 14. Nucleotide sequences encoding this amino acid sequence include the nucleotide sequence of SEQ ID NO: 6. Tropinone reductase HnTR1 of the present invention includes proteins encoded by:

(1) a polynucleotide encoding a protein comprising amino acids with one or more amino acid substitutions, deletions, insertions, and/or additions in the amino acid sequence of SEQ ID NO: 14, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 6, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 14, wherein the protein has an activity of reducing 1,1,1-trifluoroacetone represented by formula (1) to produce (S)-1,1,1-trifluoro-2-propanol represented by formula (2).

Tropinone reductase HnTR1 used in the present invention can be prepared from Hyoscyamus niger by the method described in JP-A (Kokai) 2003-230398. Alternatively, the enzyme can be obtained from a recombinant by cloning from Hyoscyamus niger or such by PCR the DNA of SEQ ID NO: 6 which encodes the Hyoscyamus niger-derived HnTR1, and then expressing HnTR1 using a recombinant prepared by introducing the DNA in an expressible form to a heterologous host, such as E. coli.

The enzyme activity of HnTR1 in the present invention can be measured, for example, as described below but it is not limited thereto. More specifically, a reaction is carried out at 30° C. in a reaction solution containing 100 mM phosphate buffer (pH 6.5), 0.2 mM NADPH, 4 mM tropinone, and the enzyme, and the decrease in absorbance at 340 nm, resulting from the decrease of NADPH is measured. 1 U is defined as the amount of enzyme that catalyzes the decrease of 1 μmol of NADH in one minute.

Next, tropinone reductase DsTR1 will be described. Tropinone reductase DsTR1 in the present invention includes proteins comprising the amino acid sequence of SEQ ID NO: 15. Nucleotide sequences encoding this amino acid sequence include the nucleotide sequence of SEQ ID NO: 7. Tropinone reductase DsTR1 of the present invention includes proteins encoded by:

(1) a polynucleotide encoding a protein comprising amino acids with one or more amino acid substitutions, deletions, insertions, and/or additions in the amino acid sequence of SEQ ID NO: 15, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 7, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 15, wherein the protein has an activity of reducing 1,1,1-trifluoroacetone represented by formula (1) to produce (S)-1,1,1-trifluoro-2-propanol represented by formula (2).

Tropinone reductase DsTR1 used in the present invention can be prepared from Datura stramonium by the method described in JP-A (Kokai) 2003-230398. Alternatively, the enzyme can be obtained from a recombinant by cloning from Datura stramonium or such by PCR the DNA of SEQ ID NO: 7 which encodes the Datura stramonium-derived DsTR1, and then expressing DsTR1 using a recombinant prepared by introducing the DNA in an expressible form to a heterologous host, such as E. coli.

The enzyme activity of DsTR1 in the present invention can be measured, for example, as described below but it is not limited thereto. More specifically, a reaction is carried out at 30° C. in a reaction solution containing 100 mM phosphate buffer (pH 6.5), 0.2 mM NADPH, 4 mM tropinone, and the enzyme, and the decrease in absorbance at 340 nm resulting from the decrease of NADPH is measured. 1 U is defined as the amount of enzyme that catalyzes the decrease of 1 μmol of NADH in one minute.

Next, alcohol dehydrogenase BstADHT will be described. Alcohol dehydrogenase BstADHT in the present invention includes proteins comprising the amino acid sequence of SEQ ID NO: 16. Nucleotide sequences encoding this amino acid sequence include the nucleotide sequence of SEQ ID NO: 8. Alcohol dehydrogenase BstADHT of the present invention includes proteins encoded by:

(1) a polynucleotide encoding a protein comprising amino acids with one or more amino acid substitutions, deletions, insertions, and/or additions in the amino acid sequence of SEQ ID NO: 16, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 8, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 16, wherein the protein has an activity of reducing 1,1,1-trifluoroacetone represented by formula (1) to produce (S)-1,1,1-trifluoro-2-propanol represented by formula (2).

Alcohol dehydrogenase BstADHT used in the present invention can be obtained from a recombinant by cloning from Geobacillus stearothermophilus or such by PCR the DNA of SEQ ID NO: 8 which encodes the Geobacillus stearothermophilus-derived BstADHT, and then expressing BstADHT using a recombinant prepared by introducing the DNA in an expressible form to a heterologous host, such as E. coli.

The enzyme activity of BstADHT in the present invention can be measured, for example, as described below but it is not limited thereto. More specifically, a reaction is carried out at 30° C. in a reaction solution containing 100 mM phosphate buffer (pH 8.0), 2.5 mM NAD⁺, 100 mM ethanol, and the enzyme, and the increase in absorbance at 340 nm resulting from the production of NADH, is measured. 1 U is defined as the amount of enzyme that catalyzes the production of 1 μmol of NADH in one minute.

Finally, alcohol dehydrogenase PfODH will be described. Alcohol dehydrogenase PfODH in the present invention includes proteins comprising the amino acid sequence of SEQ ID NO: 18. Nucleotide sequences encoding this amino acid sequence include the nucleotide sequence of SEQ ID NO: 17. Alcohol dehydrogenase PfODH of the present invention includes proteins encoded by:

(1) a polynucleotide encoding a protein comprising amino acids with one or more amino acid substitutions, deletions, insertions, and/or additions in the amino acid sequence of SEQ ID NO: 18, wherein the protein has an activity of reducing 1,1,1-trifluoroacetone represented by formula (1) to produce (S)-1,1,1-trifluoro-2-propanol represented by formula (2);
(2) a polynucleotide that hybridizes under stringent conditions with a DNA comprising the nucleotide sequence of SEQ ID NO: 17, wherein the polynucleotide encodes a protein having an activity of reducing 1,1,1-trifluoroacetone represented by formula (1) to produce (S)-1,1,1-trifluoro-2-propanol represented by formula (2); and
(3) a polynucleotide encoding a protein comprising an amino acid sequence having 80% or more homology to the amino acid sequence of SEQ ID NO: 18, wherein the protein has an activity of reducing 1,1,1-trifluoroacetone represented by formula (1) to produce (S)-1,1,1-trifluoro-2-propanol represented by formula (2).

Alcohol dehydrogenase PfODH used in the present invention can be prepared from Pichia finlandica by the method described in WO 01/061014. Alternatively, the enzyme can be obtained from a recombinant by cloning from Pichia finlandica or such by PCR the DNA of SEQ ID NO: 17 which encodes the Pichia finlandica-derived PfODH, and then expressing PfODH using a recombinant prepared by introducing the DNA in an expressible form to a heterologous host, such as E. coli.

The enzyme activity of PfODH in the present invention can be measured, for example, as described below but it is not limited thereto. More specifically, a reaction is carried out at 30° C. in a reaction solution containing 100 mM Tris-HCl buffer (pH 9.0), 2.5 mM NAD⁺, 5 mM (R)-2-octanol, and the enzyme, and the increase in absorbance at 340 nm, resulting from the production of NADH is measured. 1 U is defined as the amount of enzyme that catalyzes the production of 1 μmol of NADH in one minute.

The amino acid sequence with one or more (for example, 2 to 100, preferably 2 to 50, and more preferably 2, 3, 4, 5, 6, 7, 8, 9, or 10) amino acid deletions, substitutions, insertions, and/or additions in an above-mentioned enzyme of the present invention (a protein comprising the amino acid sequence of any one of SEQ ID NOs: 9 to 16 and 18) can be obtained by appropriately introducing a substitution, deletion, insertion, and/or addition of mutations to the polynucleotide of any one of SEQ ID NOs: 9 to 16 and 18 using site-directed mutagenesis (Nucleic Acid Res. 10, pp. 6487 (1982); Methods in Enzymol. 100, pp. 448 (1983), Molecular Cloning 2nd Ed., Cold Spring Harbor Laboratory Press (1989); PCR A Practical Approach IRL Press pp. 200 (1991)).

A polynucleotide of the present invention that can hybridize under stringent conditions with a polynucleotide comprising the nucleotide sequence of any one of SEQ ID NOs: 1 to 8 and 17 refers to a polynucleotide that hybridizes under the conditions described in the manual (for example, a wash at 42° C., primary wash buffer containing 0.5×SSC) when using, for example, ECL direct nucleic acid labeling and detection system (manufactured by Amersham Pharmacia Biotech) with a DNA prepared by selecting one or more sequences including at least 20, preferably at least 30, for example, 40, 60, or 100 arbitrary continuous nucleotides of the polynucleotide of any one of SEQ ID NOs: 1 to 8 and 17 as the probe DNA. More specifically, the term “stringent conditions” ordinarily refers to, for example, conditions of 42° C., 2×SSC, and 0.1% SDS, preferably conditions of 50° C., 2×SSC, and 0.1% SDS, and more preferably 65° C., 0.1×SSC, and 0.1% SDS, but is not particularly limited to these conditions. Multiple factors such as temperature and salt concentration can be considered as factors that affect the stringency of hybridization, and those skilled in the art can realize the most suitable stringency by appropriately selecting these factors.

Homologs of the polynucleotide in the present invention include a polynucleotide encoding a protein having homology of at least 80%, preferably at least 85%, more preferably 90%, 91%, 92%, 93%, or 94%, and even more preferably 95% or more (for example, 96%, 97%, 98%, or 99%) to the amino acid sequence of any one of SEQ ID NOs: 9 to 16 and 18. Protein homology searches can be performed, for example, against databases of amino acid sequences of proteins, such as SWISS-PROT, PIR, or DAD, databases of DNA sequences, such as DDBJ, EMBL, or Gene-Bank, or databases of amino acid sequences predicted from DNA sequences, by using programs such as BLAST and FASTA, for example, via the internet.

The form of the above-mentioned polynucleotides encoding enzymes of the present invention is not particularly limited so long as the polynucleotides can encode an enzyme of the present invention, and includes in addition to cDNA, genomic DNA and chemically synthesized DNA.

Microorganisms or transformants that functionally express enzymes, or a processed material thereof.

In the production method of the present invention, microorganisms that functionally express the above-mentioned enzymes or processed materials thereof may be used instead of the above-mentioned enzymes. Microorganisms of the present invention include isolated microorganisms expressing at least one of the above-mentioned enzymes. Without limitation, such microorganisms include, for example, Candida parapsilosis, Rhodococcus erythropolis, Streptomyces coelicolor, Zoogloea ramigera, Saccharomyces cerevisiae, Hyoscyamus niger, Datura stramonium, Geobacillus stearothermophilus, and Pichia finlandica.

Microorganisms of the present invention also include microorganisms that have been transformed with a vector comprising a DNA encoding at least one of the above-mentioned enzymes. Such microorganisms can be produced by the same method as that for producing the transformants described below.

To “functionally express” means to express an enzyme in a state in which its function is maintained. In the present invention, so long as the enzyme maintains its function, there are no limitations on the method for expressing the enzyme. Furthermore, in the present invention, the level of expression of the above-mentioned enzyme by the microorganism is also not limited. Therefore, as long as the above-mentioned enzyme is expressed, even if the amount is small, it is included in the microorganism of the present invention.

In the production methods of the present invention, instead of the above-mentioned enzyme, a transformant transformed by a vector comprising a DNA encoding the above-mentioned enzyme or a processed material thereof may be used.

A suitable vector when using a transformant transformed by a vector comprising a DNA encoding the above-mentioned enzyme, instead of the above-mentioned enzyme, includes various vectors such as plasmids, cosmids, viruses, and bacteriophages (see Molecular Cloning, A Laboratory Manual 2nd ed., Cold Spring Harbor Press (1989); Current Protocols in Molecular Biology, John Wiley & Sons (1987)). A preferred vector used in the present invention includes, for example, pK4EC prepared by inserting a gene encoding the above-mentioned enzyme of the present invention in an expressible manner to an E. coli expression vector pSE420D, but is not limited thereto.

The aforementioned vector preferably includes all components of the regulatory sequence necessary for expression of the inserted DNA. Furthermore, the vector may include a selection marker for selection of host cells introduced with the vector.

In the present invention, a sequence encoding a signal peptide can be added to the DNA. Addition of a signal peptide enables transfer of the protein expressed in the host cell to the lumen of the endoplasmic reticulum. Alternatively, when Gram-negative bacteria are used as a host, the protein expressed in the host cell can be transferred into the periplasm or transferred outside the cell using a signal peptide. One can use any signal peptide that can function in the host cell that will be used. Therefore, a signal peptide derived from a cell heterologous to the host cell can also be used. Furthermore, as necessary, a linker, a start codon (ATG), a stop codon (TAA, TAG, or TGA), and such can be added when introducing the DNA into the vector.

The microorganisms transformed to express respective enzymes in the present invention are not particularly limited so long as they are organisms that can be transformed by a recombinant vector comprising a polynucleotide encoding the respective enzyme, and can exhibit its activity. Examples of microorganisms that can be used include the following microorganisms:

Bacteria for which host-vector systems have been developed, such as:
the genus Escherichia,
the genus Bacillus,
the genus Pseudomonas,
the genus Serratia,
the genus Brevibacterium,
the genus Corynebacterium,
the genus Streptococcus, and
the genus Lactobacillus;

Actinomycetes for which host-vector systems have been developed, such as:
the genus Rhodococcus, and
the genus Streptomyces;

Yeasts for which host-vector systems have been developed, such as:
the genus Saccharomyces,
the genus Kluyveromyces,
the genus Schizosaccharomyces,
the genus Zygosaccharomyces,
the genus Yarrowia,
the genus Trichosporon,
the genus Rhodosporidium,
the genus Pichia, and
the genus Candida; and

Molds for which host-vector systems have been developed, such as:
the genus Neurospora,
the genus Aspergillus,
the genus Cephalosporium, and
the genus Trichoderma.

Procedures for production of transformants and construction of recombinant vectors compatible to the hosts can be carried out by following techniques commonly used in the field of molecular biology, bioengineering, and genetic engineering (for example, Sambrook et al., Molecular Cloning, Cold Spring Harbor Laboratories). For expression of the carbonyl reductase gene of the present invention whose electron donor is NADPH in a microorganism or such, first, the DNA is introduced into a plasmid vector or a phage vector that will stably exist in the microorganism, and then this genetic information needs to be transcribed and translated. To accomplish this, a promoter which corresponds to the regulatory unit for transcription and translation can be incorporated at the 5′-side (upstream) of the DNA strand of the present invention, and more preferably a terminator is incorporated at the 3′-side (downstream). Such promoters and terminators need to be those known to function in the microorganism to be used as the host. Such vectors, promoters, terminators, and such that can be used with various types of microorganisms are described in detail in “Biseibutsu-gaku Kiso Koza 8 (Basic Microbiological Seminar 8) Idenshi Kogaku (Genetic Engineering), Kyoritsu Shuppan”, and in particular, those that can be used with yeast are described in Adv. Biochem. Eng., 43, 75-102 (1990), and Yeast, 8, 423-488 (1992).

A variety of host and vector systems have been developed using plants and animals in addition to microorganisms. In particular, systems for expressing a large amount of heterologous proteins in insects using silkworms (Nature, 315, 592-594 (1985)) and in plants such as rapeseed, corn, or potato have been developed, and can be used suitably.

Introduction of DNA into a vector can be performed by ligase reactions using restriction enzyme sites (Current Protocols in Molecular Biology, John Wiley & Sons (1987) Section 11.4-11.11; Molecular Cloning, A Laboratory Manual 2nd ed., Cold Spring Harbor Press (1989) Section 5.61-5.63). By taking into consideration the frequency of codon usage of the host to be used, vectors with high expression efficiency can be designed by modifying the polynucleotide sequence as necessary (Grantham et al., Nucleic Acids Res. (1981) 9:r43-74).

As described above, various cells have been established as host cell strains. Methods for introducing expression vectors suitable for each cell line are also known, and those skilled in the art can select an introduction method suitable for each of the selected host cells. For example, transformation by calcium treatment, electroporation, and such are known for prokaryotic cells. Methods using agrobacterium are known for plant cells, and calcium phosphate precipitation method is an example for mammalian cells. The present invention is not particularly limited to these methods, and depending on the selected host, expression vectors can be introduced by various other known methods such as those using nuclear microinjection, protoplast fusion, DEAE-dextran method, cell fusion, electroporation, lipofectamine method (GIBCO BRL), and FuGENE6 reagent (Boehringer-Mannheim).

By culturing transformants transformed with a recombinant vector carrying the DNA as described above, proteins having the activity of reducing 1,1,1-trifluoroacetone to produce an optically active 1,1,1-trifluoro-2-propanol can be produced.

Methods for culturing the transformants are not particularly limited, and desirably, conditions such as medium, temperature, and time, which are suitable for growth of each of the selected host cell and most suitable for production of an enzyme of the present invention are selected. Enzymes can be purified from transformants by methods known to those skilled in the art. For example, transformants are grown in a medium suitable for growth of the host, and after sufficient growth, the bacterial cells are collected, the cells are homogenized in a buffer added with a reducing agent such as 2-mercaptoethanol or phenyl methane sulfonyl fluoride, or a protease inhibitor, and a cell-free extract is prepared. Enzymes can be purified from the cell-free extract by suitably combining fractionation based on solubility of the protein (precipitation by organic solvents, salting-out by ammonium sulfate, or the like), cation exchange chromatography, anion exchange chromatography, gel filtration, hydrophobic chromatography, affinity chromatography using chelates, pigments, or antibodies, or such.

In a preferred embodiment for producing optically active alcohols represented by formulas (2) and (3) described in the present invention, optically active alcohols can be produced by contacting the reaction solution with the enzyme molecule, processed material thereof, cultured material containing the enzyme molecule, or microorganisms or transformants that produce (express) the enzyme, or a processed material thereof to carry out the desired enzyme reaction. The manner in which the enzyme and the reaction solution are contacted is not limited to these specific examples.

The processed material used in the present invention refers to a product obtained by performing physical treatment, biochemical treatment, chemical treatment, or such to a biological cell. Biological cells include the above-mentioned plant cells carrying the enzyme as well as transformants that retain a gene of this enzyme in an expressible manner. Physical treatment for obtaining the processed material includes treatments such as freeze-thawing, sonication, pressurization, osmotic pressure difference, or grinding. Specific examples of biochemical treatments include treatment with a cell-wall digesting enzyme such as lysozyme. Furthermore, a chemical treatment includes treatment by contact with a surfactant, or an organic solvent such as toluene, xylene, or acetone. Processed materials include microorganisms whose cell membrane permeability has been changed by such treatment, cell-free extracts prepared by homogenizing bacterial cells by treatment with glass beads or enzymes, partially purified material thereof, or such.

Biological cells or processed materials used in the present invention can be used after immobilization by known methods such as the polyacrylamide method, sulfur-containing polysaccharide gel method (such as K-carrageenan gel method), alginic acid gel method, agar gel method, or ion exchange resin method.

### Coenzymes

A preferred embodiment of the present invention provides methods for producing (S)-1,1,1-trifluoro-2-propanol (hereinafter abbreviated as (S)-TFIP) by reacting a protein having carbonyl reductase activity, which is selected from CpSADH, ReSADH, ScoPAR, ZraSBDH, ScGCY1, HnTR1, DsTR1, or BstADHT, a microorganism or a transformant that produces (expresses) the enzyme or protein, or a processed material thereof with 1,1,1-trifluoroacetone (hereinafter abbreviated as TFAC). Furthermore, the present invention provides methods for producing (R)-1,1,1-trifluoro-2-propanol (hereinafter abbreviated as (R)-TFIP) by reacting a protein having carbonyl reductase activity (PfODH), a microorganism or a transformant that produces (expresses) the enzyme or protein, or a processed material thereof with TFAC.

In the production methods of the present invention, in addition to the protein (enzyme) having carbonyl reductase activity, a coenzyme corresponding to this enzyme, and a dehydrogenase having the activity to regenerate reduced nicotinamide adenine dinucleotide (NADH) or reduced nicotinamide adenine dinucleotide phosphate (NADPH) may be used simultaneously.

Regeneration of NAD(P)H from NAD(P)⁺ produced from NAD(P)H accompanying the above-mentioned reduction reaction, can be achieved by using the ability of a microorganism to reduce NAD(P)⁺ (glycolytic pathway, the pathway of methylotrophs to assimilate C1 compounds, etc.). The ability to reduce NAD(P)⁺ can be enhanced by adding glucose, ethanol, or such to the reaction system. The reduction reaction can be achieved by adding either a microorganism having the ability to produce NAD(P)H from NAD(P)⁺, or a processed material thereof or enzyme thereof, to the reaction system. For example, NAD(P)H can be regenerated using a microorganism containing glucose dehydrogenase, alcohol dehydrogenase, formate dehydrogenase, amino acid dehydrogenase, organic acid dehydrogenase (such as malate dehydrogenase), phosphite dehydrogenase, hydrogenase, or such, a processed material thereof, or a purified or partially purified enzyme. Such components constituting the required reactions for NAD(P)H regeneration may either be added to the reaction system to produce an optically active alcohol according to the present invention, added to the system after being immobilized, or can be contacted with the system via an NAD(P)H-exchangeable membrane.

Herein, the combination of an enzyme having carbonyl reductase activity and a dehydrogenase having the activity to regenerate reduced nicotinamide adenine dinucleotide (NADH) or reduced nicotinamide adenine dinucleotide phosphate (NADPH) used in the present invention is not limited, but a preferred combination includes a combination of a dehydrogenase selected from the dehydrogenases described below in (1) and an enzyme having carbonyl reductase activity selected from enzymes having carbonyl reductase activity described below in (2). More specifically, for example, when CpSADH is selected as the enzyme having carbonyl reductase activity, the dehydrogenase used in combination may be a dehydrogenase selected from any of glucose dehydrogenase, alcohol dehydrogenase, formate dehydrogenase, amino acid dehydrogenase, organic acid dehydrogenase (such as malate dehydrogenase), phosphite dehydrogenase, or hydrogenase. This is the same when an enzyme other than CpSADH is selected as the enzyme having carbonyl reductase activity. Accordingly, in the method for producing optically active alcohols of the present invention, the combination of dehydrogenase and an enzyme having carbonyl reductase activity is not limited to those described in the Examples.

### (1) Dehydrogenases

glucose dehydrogenase, alcohol dehydrogenase, formate dehydrogenase, amino acid dehydrogenase, organic acid dehydrogenase (such as malate dehydrogenase), phosphite dehydrogenase, hydrogenase
(2) Enzymes Having Carbonyl Reductase Activity alcohol dehydrogenase CpSADH, alcohol dehydrogenase ReSADH, carbonyl reductase ScoPAR, (2S,3S)-butanediol dehydrogenase ZraSBDH, carbonyl reductase ScGCY1, tropinone reductase HnTR1, tropinone reductase DsTR1, alcohol dehydrogenase BstADHT, alcohol dehydrogenase PfODH

Furthermore, a method of the present invention may include the step of culturing a transformant transformed with a recombinant vector comprising a polynucleotide encoding a polypeptide that can be used in the present invention. In the method of the present invention, when using live microbial cells transformed with a recombinant vector comprising a polynucleotide of the present invention, there are cases when an additional reaction system for NAD(P)H regeneration is not necessary. More specifically, by using as the host a microorganism with a high level of NAD(P)H regeneration activity, an efficient reaction can be carried out without supplementing an enzyme for NAD(P)H regeneration to the reduction reaction using the transformant. Furthermore, by simultaneously introducing to a host a DNA encoding an NAD(P)H-dependent enzyme of the present invention and a gene for glucose dehydrogenase, alcohol dehydrogenase, formate dehydrogenase, amino acid dehydrogenase, organic acid dehydrogenase (such as malate dehydrogenase), phosphite dehydrogenase, hydrogenase, or such usable for NAD(P)H regeneration, a more efficient expression of the NAD(P)H-dependent carbonyl reductase and reduction reaction can be carried out. To introduce these two or more genes into the host, a method for transforming a host with recombinant vectors in which a plurality of vectors with different replication origins are separately introduced with genes, a method for introducing both genes into a single vector, or a method for introducing both or one of the genes into a chromosome can be used to avoid incompatibility.

When introducing a plurality of genes into a single vector, a method is used in which regions relating to the regulation of expression, such as the promoter and terminator, are linked to each gene, or alternatively the genes may be expressed as an operon containing multiple cistrons, such as the lactose operon.

For example, glucose dehydrogenases derived from Bacillus subtilis and Thermoplasma acidophilum can be used as the enzymes for NADPH regeneration. Furthermore, formate dehydrogenase derived from Mycobacterium vaccae can be used as the enzyme for NADH regeneration.

More specifically, for the synthesis of optically active TFIP, a coexpression plasmid introduced with both a gene of an enzyme having the ability to reduce a carbonyl, which is any one of CpSADH, ReSADH, ScoPAR, ZraSBDH, and PfODH, and a Mycobacterium vaccae-derived formate dehydrogenase gene (pSF-CPA4 (described in the Examples of the present invention), pSF-RED1 (described in the Examples of the present invention), pSF-SCP7 (described in the Examples of the present invention), pSF-ZRD1 (JP-A (Kokai) 2004-357639), and pSF-PFO2 (WO 01-061014) respectively) can be used. Another example is a coexpression plasmid introduced with both a gene of an enzyme having the ability to reduce a carbonyl, which is any one of ScGCY1, HnTR1, and DsTR1, and a Bacillus subtilis-derived glucose dehydrogenase gene (pSG-GCY1 (described in the Examples of the present invention), pSG-HNR1 (JP-A (Kokai) 2003-230398), and pSG-DSR1 (JP-A (Kokai) 2003-230398)).

The reduction reaction using an enzyme of the present invention can be carried out in water, in an organic solvent that is poorly soluble in water, for example, organic solvents such as ethyl acetate, butyl acetate, toluene, chloroform, hexane, methyl isobutyl ketone, or methyl tertiary butyl ester; a two-phase system with an aqueous medium; or a mixed system with an organic solvent soluble in water, for example, methanol, ethanol, isopropyl alcohol, acetonitrile, acetone, or dimethylsulfoxide. The reaction of the present invention can also be carried out using immobilized enzymes, membrane reactors, and the like.

Furthermore, there is no limitation on the concentration of TFAC represented by formula (1), which is the raw material of the present reaction. The reaction is usually performed at a substrate concentration of 0.01% to 50%, preferably at 0.1% to 20%, and more preferably at 1% to 10%. The substrate can be added either at once at the start of the reaction, or continuously or intermittently to prevent the substrate concentration in the reaction solution from becoming too high. The substrate can be placed in the reaction system in the form of TFAC represented by formula (1), but it may also be added in the form of the hydrate represented by formula (4), or as an aqueous solution of the compound represented by formula (1) or (4), or in the form of a solution in other solvents.

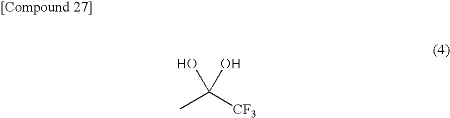

In the present invention, each of “concentration %” means “weight of the raw material or product/weight of the reaction solution (w/w) %” and “conversion rate” means “concentration of the product/([concentration of the remaining raw material]+[concentration of the product]) %”. In the case of (S)-TFIP, % e.e. means “([(S)-TFIP concentration]−[(R)-TFIP concentration])/([(S)-TFIP concentration]+[(R)-TFIP concentration])×100”. Similarly, in the case of (R)-TFIP, % e.e. means “([(R)-TFIP concentration]−[(S)-TFIP concentration])/([(R)-TFIP concentration]+[(S)-TFIP concentration])×100”.

For reactions of the present invention, a temperature at which the enzyme can exhibit its catalytic activity can be selected. More specifically, the reaction can be carried out at a reaction temperature of 4° C. to 50° C., preferably 10° C. to 40° C., and more preferably 10° C. to 30° C. Since the reaction substrate, TFAC, represented by formula (1) and the products, optically active TFIPs, represented by formulas (2) and (3) of the present invention all have low boiling points, the reaction is desirably carried out at low temperature. For the reaction pH, a range in which the enzyme can exhibit its catalytic activity can be selected. More specifically, the reaction can be carried out at pH 3 to 9, at pH 4 to 8, and more preferably at pH 5 to 7. Furthermore, a coenzyme, NAD⁺, NADH, NADP⁺, or NADPH, may be added as necessary to the reaction system at 0.001 mM to 100 mM, or preferably 0.01 mM to 10 mM.

To regenerate NADH and NADPH, for example, glucose is added to the reaction system when glucose dehydrogenase is used, ethanol or isopropanol is added when alcohol dehydrogenase is used, formic acid is added when formate dehydrogenase is used, amino acid is added when amino acid dehydrogenase is used, malic acid is added when malate dehydrogenase is used, glycerol is added when glycerol dehydrogenase is used, phosphorous acid is added when phosphite dehydrogenase is used, or hydrogen is added when hydrogenase is used. Such a compound may be added at a molar ratio of 0.1 to 20, or preferably 1 to 5-fold excess to the substrate ketone. On the other hand, an enzyme for NAD(P)H regeneration, such as glucose dehydrogenase, alcohol dehydrogenase, formate dehydrogenase, amino acid dehydrogenase, malate dehydrogenase, glycerol dehydrogenase, phosphite dehydrogenase, or hydrogenase, may be added at enzymatic activity 0.01 to 100 times higher, or preferably about 0.1 to 20 times higher than that of the NAD(P)H-dependent enzyme of the present invention.

Furthermore, the production method of the present invention may also include the step of collecting (S)-TFIP or (R)-RFIP. (S)-TFIP or (R)-RFIP can be collected, for example, by the method described in the Examples, but is not limited thereto.

Optically active TFIP produced by reducing TFAC according to the present invention can be purified by a suitable combination of centrifugation of bacterial cells and proteins, separation by membrane treatment, solvent extraction, distillation, drying using a drying agent, column chromatography, and such.

For example, in optically active TFIP synthesis, optically active TFIP can be obtained by distilling the reaction solution containing the microbial cells. Preferably, before the distillation procedure, a procedure to remove the bacterial cells, for example, centrifugation, membrane treatment, and such can be performed. Furthermore, for higher purity of the reaction product and especially to decrease the water content, the reaction product is dried using various types of drying agents, and the product can be purified to a higher degree by redistillation when necessary. Drying agents generally used for drying can be used.

Optically active TFIP means TFIP under a condition in which the concentration of one of the enantiomers is higher than the concentration of the other enantiomer. High optical purity sufficient for industrial use includes 90% e.e. or more, and preferably 93% e.e. or more.

All prior art references cited herein are incorporated herein by reference.

### EXAMPLES

The present invention is illustrated in detail below with reference to Examples, but is not to be construed as being limited thereto.

Herein, an LB medium refers to a medium containing 1% Bacto Tryptone, 0.5% Bacto Yeast Extract, 1% sodium chloride, and having a pH of 7.2, and an ampicillin-containing LB medium refers to a medium prepared by adding ampicillin to the LB medium with the above composition at a concentration of 50 mg/mL.

### Measurement of Enzyme Activities

Escherichia coli HB101 strain or JM109 strain was transformed with the plasmids prepared in the Examples described below and other plasmids described in the references. The resultant transformants were cultured in ampicillin-containing LB medium overnight. 0.1 mM isopropyl-1-thio-β-D-galactopyranoside (hereinafter abbreviated as IPTG) was added to induce the expression of genes, and the cultivation was further continued for four hours. The resultant bacterial cells were collected, suspended in tris-hydrochloride or phosphate buffer solution containing 0.02% mercaptoethanol, and lysed with a closed system ultrasonic cell disrupter UCD-200™ (Cosmo Bio Co., Ltd.). The lysate was centrifuged, and the supernatant was used as a cell-free extract. Using the cell-free extract, enzyme activities were measured by the following methods.

### (Measurement of CpSADH, ReSADH, ScoPAR, and PfODH Activities)

A reaction solution containing the cell-free extract, 100 mM tris-hydrochloride buffer solution (pH 9.0), a substrate, and 2.5 mM NAD⁺ was allowed to react at 30° C., and the increase in absorbance at 340 nm, resulting from NADH production, was measured. 1 U was defined as the amount of enzyme capable of catalyzing the production of 1 μmol of NADH in one minute. As substrates, 50 mM (S)-1,3-butanediol was used to measure the activity of CpSADH, 5 mM (S)-2-octanol was used to measure the activity of ReSADH and ScoPAR, and 5 mM (R)-2-octanol was used to measure the activity of PfODH.

### (Measurement of ZraSBDH, BstADHT, ScADH1, and ScADH2 Activities)

A reaction solution containing the cell-free extract, 100 mM phosphate buffer solution (pH 8.0), a substrate, and 2.5 mM NAD⁺ was allowed to react at 30° C., and the increase in absorbance at 340 nm, resulting from NADH production was measured. 1 U was defined as the amount of enzyme capable of catalyzing the production of 1 μmol of NADH in one minute. As substrates, 50 mM (2S,3S)-butanediol was used to measure the activity of ZraSBDH, and 100 mM ethanol was used to measure the activity of BstADHT, ScADH1, and ScADH2.

### (Measurement of ScGCY1, HnTR1, DsTR1, and ScGRE3 Activities)

A reaction solution containing the cell-free extract, 100 mM phosphate buffer solution (pH 6.5), a substrate, and 0.2 mM NADPH was allowed to react at 30° C., and the decrease in absorbance at 340 nm, which results from the decrease of NADPH, was measured. 1 U was defined as the amount of enzyme capable of catalyzing the decrease of 1 μmol of NADPH in one minute. As substrates, 20 mM ethyl acetoacetate was used to measure the activity of ScGCY1, 4 mM tropinone was used to measure the activity of HnTR1 and DsTR1, and 100 mM xylose was used to measure the activity of ScGRE3.

### (Measurement of Formate Dehydrogenase Activity)

A reaction solution containing the cell-free extract, 100 mM phosphate buffer solution (pH 7.0), 100 mM sodium formate, and 2.5 mM NAD⁺ was allowed to react at 30° C., and the increase in absorbance at 340 nm, resulting from NADH production, was measured. 1 U was defined as the amount of enzyme capable of catalyzing the production of 1 μmol of NADH in one minute.

### (Measurement of Glucose Dehydrogenase Activity)

A reaction solution containing the cell-free extract, 100 mM phosphate buffer solution (pH 7.0), 100 mM D-glucose, and 2.5 mM NAD⁺ was allowed to react at 30° C., and the increase in absorbance at 340 nm, resulting from NADH production, was measured. 1 U was defined as the amount of enzyme capable of catalyzing the production of 1 μmol of NADPH in one minute.

### Analysis Conditions

### (Analysis Condition 1) Quantitative Analysis of TFAC and TFIP

Organic layers prepared by extracting each of the reaction solutions with an equal volume of dichloromethane, or samples prepared by dissolving 10 mg of TFIP in 1 mL of acetonitrile were analyzed under the following conditions.

Column: Supelcowax 10 (Supelco) (30 m×0.25 mm×0.25 μm)

Column temperature: 60° C. to 120° C. (4° C./minute)

Inlet and detector temperature: 250° C.

Detection method: hydrogen flame ionization

Carrier gas: helium (100 kPa)

As a result of the analysis under the conditions above, TFAC and TFIP were detected at retention times of 2.2 minutes and 5.6 minutes, respectively.

### (Analysis Condition 2) Optical Purity Analysis of TFIP

Organic layers prepared by extracting each of the reaction solutions with an equal volume of dichloromethane, or samples prepared by dissolving 5 mg of TFIP in 1 mL of dichloromethane were analyzed under the following conditions.

Column: BGB-174 (BGB Analytik) (30 m×0.25 mm×0.25 μm)

Column temperature: 60° C. to 85° C. (1° C./minute) to 110° C. (5° C./minute)

Inlet temperature: 180° C.

Detector temperature: 200° C.

Detection method: hydrogen flame ionization

Carrier gas: helium (100 kPa)

As a result of the analysis under the conditions above, (R)-TFAC and (S)-TFIP were detected at retention times of 21.8 minutes and 23.5 minutes, respectively.

### Example 1

### Construction of Plasmid pSF-CPA4 Coexpressing Alcohol Dehydrogenase CpSADH Gene Derived from Candida parapsilosis and Formate Dehydrogenase McFDH Gene Derived from Mycobacterium vaccae

A sense primer CPA-ATG5 (SEQ ID NO: 19) and an antisense primer CPA-TAA5 (SEQ ID NO: 20) were synthesized for cloning based on the nucleotide sequence (Accession No. E09871) described in Japanese Patent No. 3574682.

SEQ ID NO: 19

GTGGAATTCTATAATGTCAATTCCATCAAGCCAG

SEQ ID NO: 20

CTGAAGCTTATTATGGATTAAAAACAACACGACCTTCATAAGC

50 μL of a mixture containing 10 pmol each of the primers CPA-ATG5 and CPA-TAA5, 10 pmol of dNTP, 10 pmol of the plasmid pSE-CPA1 described in Biosci. Biotechnol. Biochem., 66, 481-483 (2002), and 1.25 U of Pfu Turbo DNA polymerase (STRATAGENE) was subjected to 30 PCR cycles of denaturation at 95° C. for 30 seconds, annealing at 50° C. for 1 minute, and extension at 72° C. for 2 minutes 30 seconds using GeneAmp PCR System 2400, thereby obtaining a specific amplification product.

The amplified product was double-digested with restriction enzymes NcoI and XbaI, and then electrophoresed in an agarose gel. The band of interest was cut out, and then purified using GFX PCR DNA and Gel Band Purification Kit (Pharmacia).

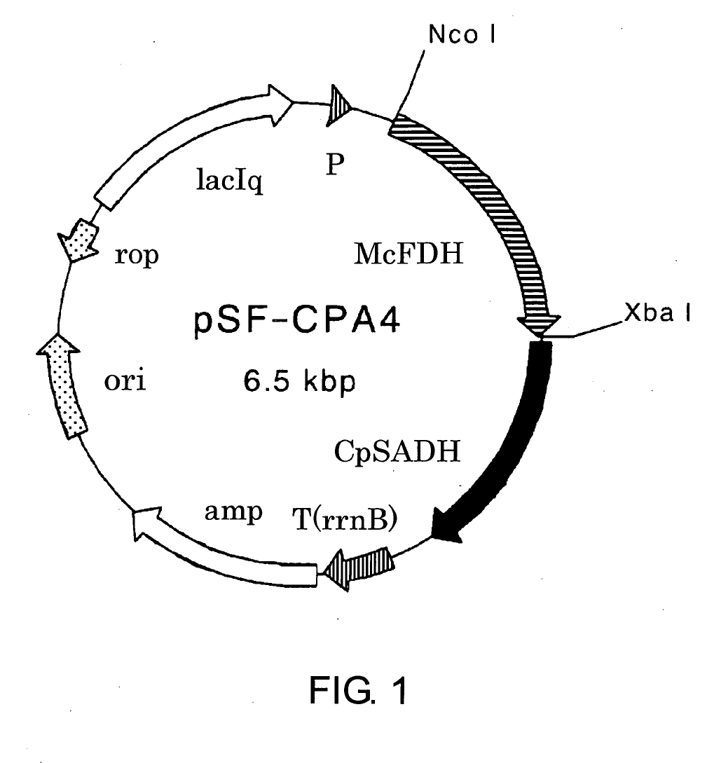

The resultant PCR-amplified DNA fragments after digestion with the restriction enzymes were ligated with a vector pSE-MF26 (JP-A (Kokai) 2003-199595), which had been double-digested with restriction enzymes NcoI and XbaI using TAKARA Ligation Kit. Escherichia coli JM109 strain was transformed with the ligated DNA. The transformant was grown in ampicillin-containing LB medium, and then a plasmid was purified from the transformant using FlexiPrep Kit (Pharmacia). The nucleotide sequence of the DNA insert of the purified plasmid was analyzed, and the determined nucleotide sequence and its amino acid sequence are indicated by SEQ ID NO: 1 and SEQ ID NO: 9, respectively. The plasmid obtained was named pSF-CPA4 (FIG. 1).

### Example 2

### Enzyme Activity of the Transformant Transformed with Plasmid Coexpressing Alcohol Dehydrogenase CpSADH Derived from Candida parapsilosis and Formate Dehydrogenase McFDH Derived from Mycobacterium vaccae

A cell-free extract was prepared according to the above-described method using the Escherichia coli HB101 strain transformed with pSF-CPA4 obtained in Example 1. The enzyme activity was measured according to the above-described method. The specific activities of CpSADH and McFDH were 5.96 U/mg protein and 0.474 U/mg protein, respectively.

### Example 3

### Preparation of Chromosomal DNA from Rhodococcus erythropolis

Rhodococcus erythropolis DSM 743 strain was cultured in a broth medium, and bacterial cells were prepared. Preparation of chromosomal DNA from the bacterial cells was performed by the method described in Nucleic Acids Res., 8, 4321 (1980).

### Example 4

### Cloning of Alcohol Dehydrogenase ReSADH Derived from Rhodococcus erythropolis

A sense primer RE-ATG1 (SEQ ID NO: 21) and an antisense primer RE-TAA1 (SEQ ID NO: 22) were synthesized for cloning based on the nucleotide sequence (Accession No. AY161280) described in Appl. Microbiol. Biotechnol., 62, 380-386 (2003).

SEQ ID NO: 21

CACGAATTCTATCATGAAAGCAATCCAGTACACG

SEQ ID NO: 22

TCGAAGCTTCTAGATTAAAGACCAGGGACCACAAC

50 μL of a mixture containing 10 pmol each of the primers RE-ATG1 and RE-TAA1, 10 nmol of dNTP, 50 ng of the Rhodococcus erythropolis-derived chromosome prepared in Example 3, and 1.25 U of Pfu Turbo DNA polymerase (STRATAGENE) was subjected to 30 PCR cycles of denaturation at 95° C. for 30 seconds, annealing at 50° C. for 1 minute, and extension at 72° C. for 2 minutes 30 seconds using GeneAmp PCR System 2400, thereby obtaining a specific amplification product.

The amplified product was double-digested with restriction enzymes EcoRI and HindIII, and then electrophoresed in an agarose gel. The band of interest was cut out, and then purified using GFX PCR DNA and Gel Band Purification Kit (Pharmacia).

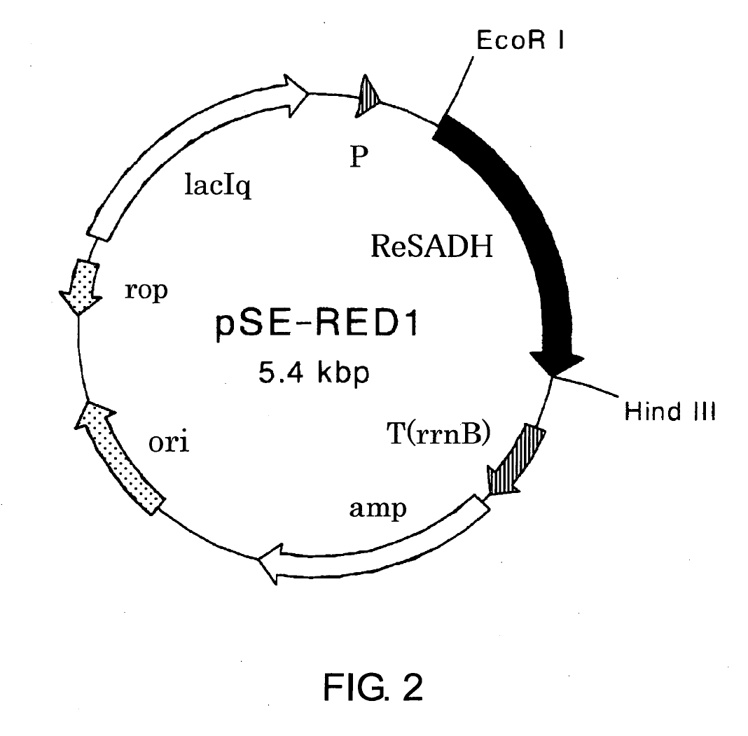

The resultant PCR-amplified DNA fragments after digestion with the restriction enzymes were ligated with a vector pSE420D (JP-A (Kokai) 2000-189170), which had been double-digested with restriction enzymes EcoRI and Hind III using TAKARA Ligation Kit. Escherichia coli JM109 strain was transformed with the ligated DNA. The transformant was grown in an ampicillin-containing LB medium, and then a plasmid was purified from the transformant using FlexiPrep Kit (Pharmacia). The nucleotide sequence of the DNA insert of the purified plasmid was analyzed and compared with the nucleotide sequence described in the reference; except for the primer region, substitutions of four bases and one amino acid were found. The determined nucleotide sequence and its amino acid sequence are indicated by SEQ ID NO: 2 and SEQ ID NO: 10, respectively. The plasmid obtained was named pSE-RED1 (FIG. 2).

### Example 5

### Construction of Plasmid pSF-RED1 Coexpressing Alcohol Dehydrogenase ReSADH Gene Derived from Rhodococcus erythropolis and Formate Dehydrogenase McFDH Gene Derived from Mycobacterium vaccae

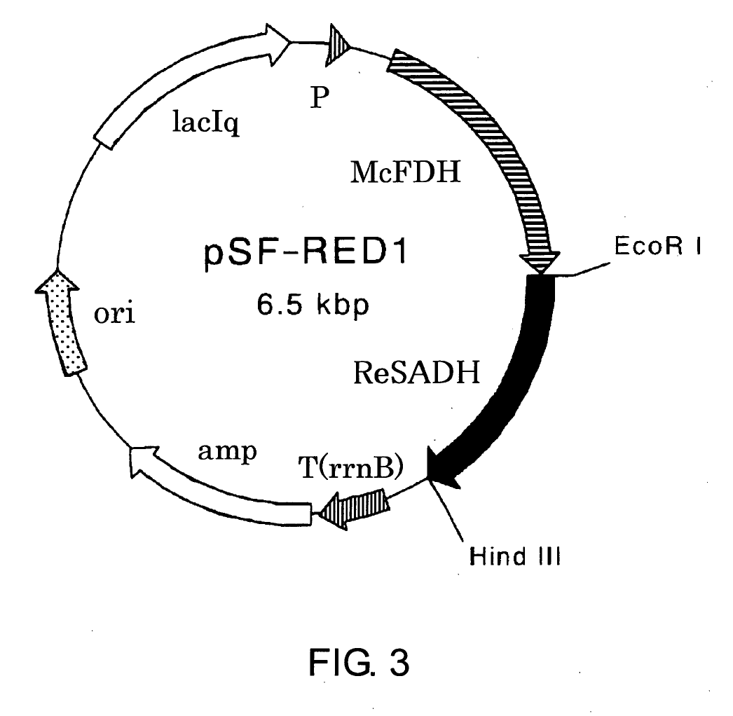

pSE-RED1 obtained in Example 4 was double-digested with restriction enzymes EcoRI and HindIII, and the resultant DNA fragments were purified. Further, plasmid pSE-MF26 (JP-A (Kokai) 2003-199595) containing a formate dehydrogenase gene derived from Mycobacterium vaccae was double-digested with restriction enzymes EcoRI and HindIII, and ligated with the DNA fragment cut out from pSL-RED1 using TAKARA Ligation Kit. Escherichia coli JM109 strain was transformed with the ligated DNA. The transformant was grown in an ampicillin-containing LB medium, and then a plasmid was purified from the resultant transformant using FlexiPrep Kit (Pharmacia) to obtain the plasmid pSF-RED1 (FIG. 3) capable of coexpressing ReSADH and formate dehydrogenase McFDH.

### Example 6

### Enzyme Activity of the Transformant Transformed with a Plasmid Coexpressing Alcohol Dehydrogenase ReSADH Derived from Rhodococcus erythropolis and Formate Dehydrogenase McFDH Derived from Mycobacterium vaccae

A cell-free extract was prepared according to the above-described method using the Escherichia coli HB101 strain transformed with pSF-RED1 obtained in Example 5. The enzyme activity was measured according to the above-described method. The specific activities of ReSADH and McFDH were 8.49 U/mg protein and 1.52 U/mg protein, respectively.

### Example 7

### Cloning of Carbonyl Reductase ScoPAR Derived from Streptomyces coelicolor

DNA was synthesized based on the amino acid sequence of SEQ ID NO: 11. The sequence of the synthetic DNA is indicated by SEQ ID NO: 3. The resultant sequence was double-digested with restriction enzymes EcoRI and HindIII, and then purified.

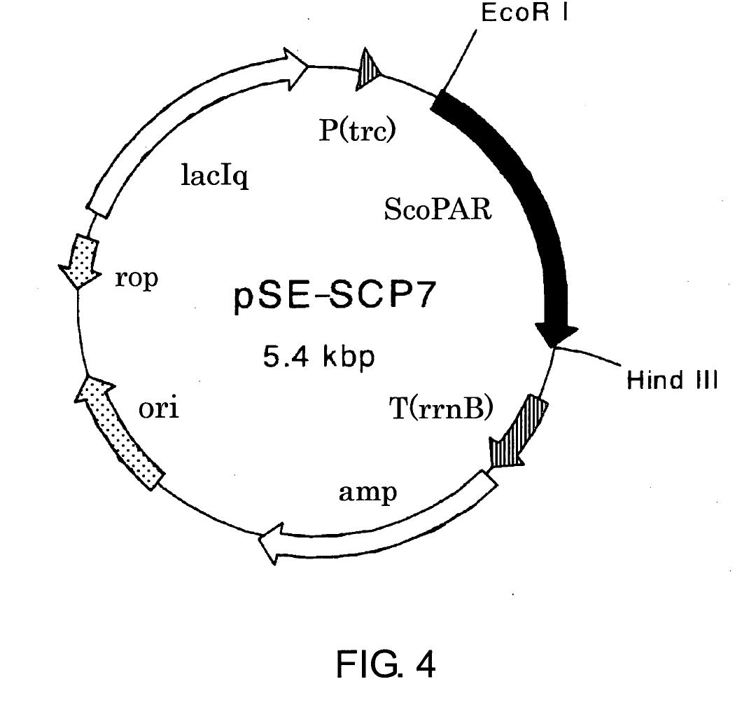

The resultant synthetic DNA fragments after digestion with the restriction enzymes were ligated with the vector pSE420D (JP-A (Kokai) 2000-189170), which had been double-digested with restriction enzymes EcoRI and HindIII, using TAKARA Ligation Kit. Escherichia coli JM109 strain was transformed with the ligated DNA. The transformant was grown in an ampicillin-containing LB medium, and a plasmid was purified from the resultant transformant using FlexiPrep Kit (Pharmacia). The nucleotide sequence of the DNA insert of the purified plasmid was analyzed, and found to be consistent with the nucleotide sequence of SEQ ID NO: 11. The plasmid obtained was named pSE-SCP7 (FIG. 4).

### Example 8

### Construction of Plasmid pSF-SCP7 Coexpressing Carbonyl Reductase ScoPAR Derived from Streptomyces coelicolor and Formate Dehydrogenase McFDH Gene Derived from Mycobacterium vaccae

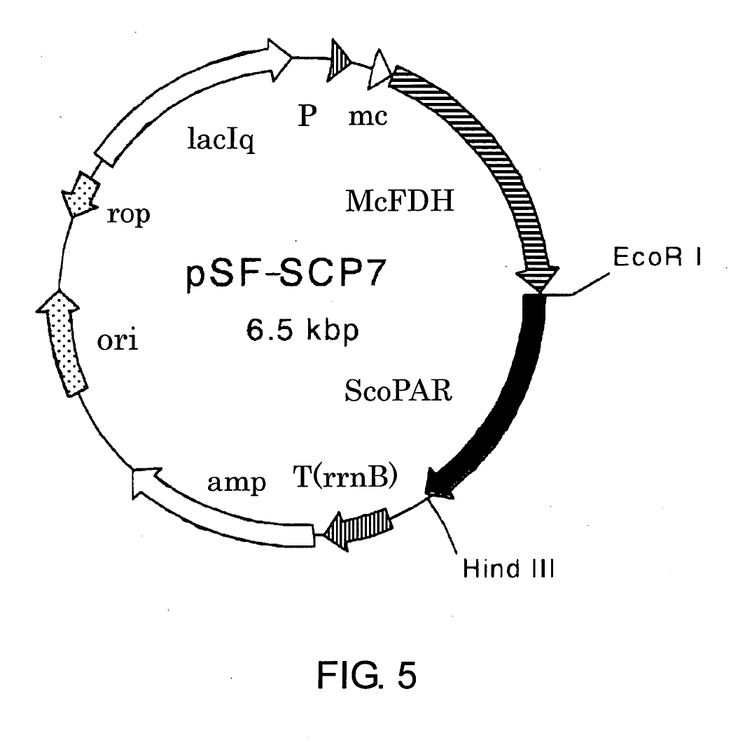

pSE-SCP7 obtained in Example 7 was double-digested with restriction enzymes EcoRI and HindIII, and the resultant DNA fragments were purified. Further, the plasmid pSU-MF26 (Japanese Patent Application No. 2005-169919) containing a formate dehydrogenase McFDH gene derived from Mycobacterium vaccae was double-digested with restriction enzymes EcoRI and HindIII, and ligated with DNA fragments cut out from the pSL-SCP7. Escherichia coli JM109 strain was transformed with the ligated DNA. The transformant was grown in an ampicillin-containing LB medium, and a plasmid was purified from the resultant transformant using FlexiPrep Kit (Pharmacia) to obtain the plasmid pSF-SCP7 (FIG. 5) capable of coexpressing ScoPAR and McFDH.

### Example 9

### Enzyme Activity of the Transformant Transformed with a Plasmid Coexpressing Carbonyl Reductase ScoPAR Derived from Streptomyces coelicolor and Formate Dehydrogenase McFDH Derived from Mycobacterium vaccae

A cell-free extract was prepared according to the above-described method using the Escherichia coli HB101 strain transformed with pSF-SCP7 obtained in Example 8. The enzyme activity was measured according to the above-described method. The specific activities of ScoPAR and McFDH were 0.67 mU/mg protein and 0.25 U/mg protein, respectively.

### Example 10

### Preparation of Chromosomal DNA from Saccharomyces cerevisiae

Saccharomyces cerevisiae was cultured in a YEPD medium, and bacterial cells were prepared. Preparation of chromosomal DNA from the bacterial cells was performed by the method described in Meth. Cell Biol., 29, 39 (1975).

### Example 11

### Cloning of Carbonyl Reductase ScGCY1 Derived from Saccharomyces cerevisiae

A sense primer GCY1-ATG1 (SEQ ID NO: 23) and an antisense primer GCY1-TAA1 (SEQ ID NO: 24) were synthesized for cloning based on the nucleotide sequence (Accession No. X13228) of the carbonyl reductase gene derived from Saccharomyces cerevisiae described in FEBS Lett., 238, 123-128 (1988).

SEQ ID NO: 23

GAGCCATGGCACCTGCTACTTTACATGATTCTACGAA

SEQ ID NO: 24

GAGCTTAAGTCTAGATTATTTGAATACTTCGAAAGGAGACCA

Using the chromosomal DNA obtained in Example 10, which had been purified from Saccharomyces cerevisiae, as the template, PCR was carried out in the same manner as in Example 4 using the primers GCY1-ATG1 and GCY1-TAA1.

The amplified product was double-digested with restriction enzymes NcoI and XbaI, and then electrophoresed in an agarose gel. The band of interest was cut out, and then purified using GFX PCR DNA and Gel Band Purification Kit (Pharmacia).

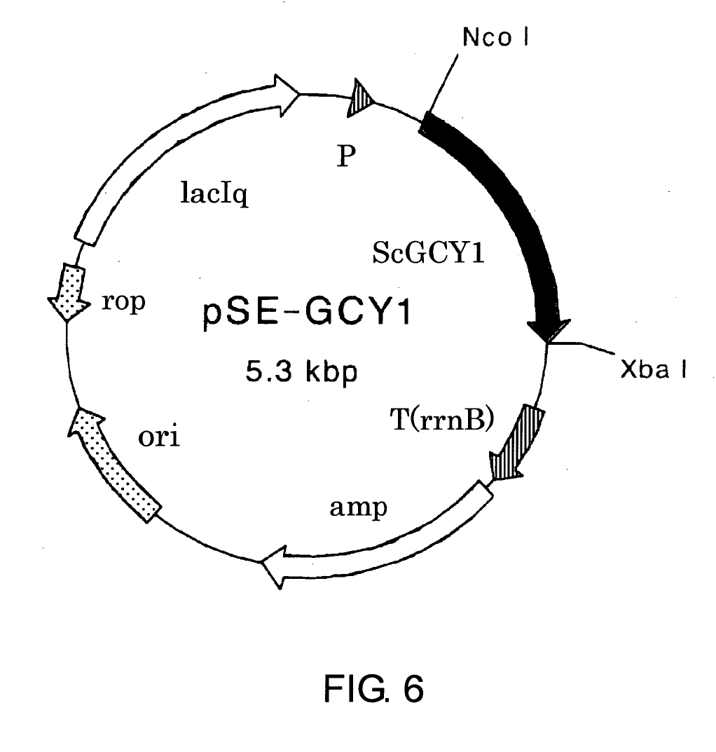

The resultant PCR-amplified DNA fragments after digestion with the restriction enzymes were ligated with a vector pSE420D (JP-A (Kokai) 2000-189170), which had been double-digested with restriction enzymes NcoI and XbaI. Escherichia coli JM109 strain was transformed with the ligated DNA. The transformant was grown in an ampicillin-containing LB medium, and a plasmid was purified from the resultant transformant using FlexiPrep Kit (Pharmacia). The nucleotide sequence of the DNA insert of the purified plasmid was analyzed, and found to be consistent with the nucleotide sequence described in the reference, except for the primer region. The determined nucleotide sequence and its amino acid sequence are indicated by SEQ ID NO: 5 and SEQ ID NO: 13, respectively. The plasmid obtained was named pSE-GCY1 (FIG. 6).

### Example 12

### Construction of Plasmid pSG-GCY1xpressing Carbonyl Reductase ScGCY1 Derived from Saccharomyces cerevisiae and Glucose Dehydrogenase BsGDH Gene Derived from Bacillus subtilis

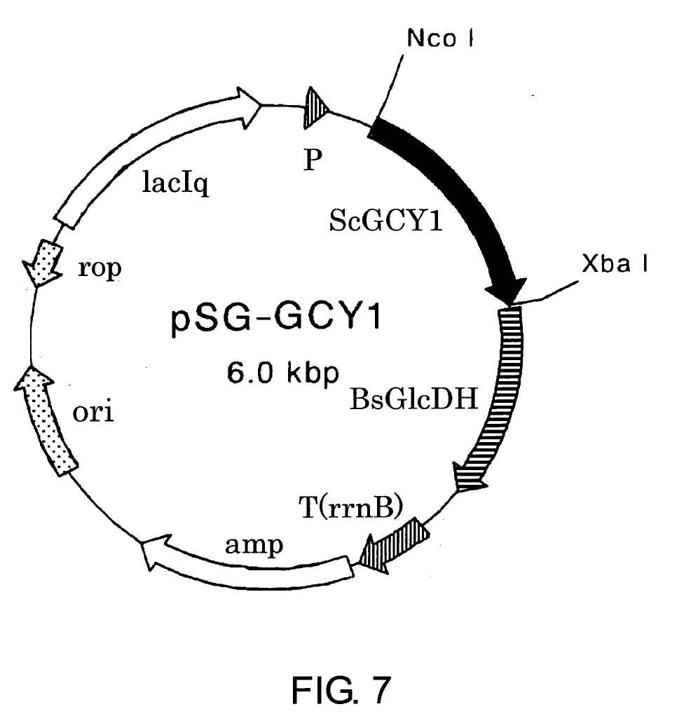

pSE-GCY1 obtained in Example 11 was double-digested with restriction enzymes NcoI and XbaI, and the resultant DNA fragments were purified. Further, the plasmid pSE-BSG1 (JP-A (Kokai) 2000-189170) containing glucose dehydrogenase BsGDH gene derived from Bacillus subtilis was double-digested with restriction enzymes NcoI and XbaI, and then ligated with DNA fragments cut out from the pSL-GCY1. Escherichia coli JM109 strain was transformed with the ligated DNA. The transformant was grown in an ampicillin-containing LB medium, and a plasmid was purified from the resultant transformant using FlexiPrep Kit (Pharmacia) to obtain a plasmid pSG-GCY1 capable of coexpressing ScGCY1 and BsGDH (FIG. 7).

### Example 13

### Enzyme Activity of the Transformant Transformed with a Plasmid Coexpressing Carbonyl Reductase ScGCY1 Derived from Saccharomyces cerevisiae and Glucose Dehydrogenase BsGDH Derived from Bacillus subtilis

A cell-free extract was prepared according to the above-described method using the Escherichia coli HB101 strain transformed with pSF-GCY1 obtained in Example 12. The enzyme activity was measured according to the above-described method. The specific activities of ScGCY1 and BsGDH were 0.27 U/mg protein and 3.72 U/mg protein, respectively.

### Example 14

### Cloning of Alcohol Dehydrogenase BstADHT Derived from Geobacillus stearothermophilus

A sense primer BSAT-AT1 (SEQ ID NO: 25) and an antisense primer BSAT-TA1 (SEQ ID NO: 26) were synthesized for cloning based on the nucleotide sequence (Accession No. D90421) of an alcohol dehydrogenase gene derived from Geobacillus stearothermophilus described in J. Bacteriol., 174, 1397-1402 (1992).

SEQ ID NO: 25

GAGGAATTCAATCATGAAAGCTGCAGTTGTG

SEQ ID NO: 26

GTCAAGCTTCTAGATTAATCTACTTTTAACACGACGC

Using the plasmid pTBAD40 as the template, PCR was carried out in the same manner as in Example 4 using the primers BSAT-AT1 and BSAT-TA1.

The amplified product was double-digested with restriction enzymes BspHI and XbaI, and electrophoresed in an agarose gel. The band of interest was cut out, and then purified using GFX PCR DNA and Gel Band Purification Kit (Pharmacia).

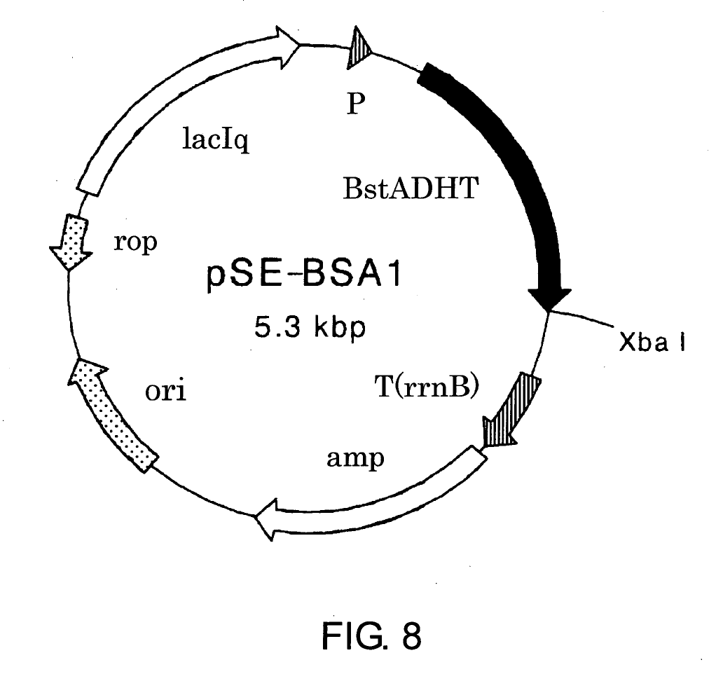

The resultant PCR-amplified DNA fragments after digestion with the restriction enzymes were ligated with a vector pSE420D (JP-A (Kokai) 2000-189170), which had been double-digested with restriction enzymes NcoI and XbaI. Escherichia coli JM109 strain was transformed with the ligated DNA. The transformant was grown in an ampicillin-containing LB medium, and a plasmid was purified from the resultant transformant using FlexiPrep Kit (Pharmacia). The nucleotide sequence of the DNA insert of the purified plasmid was analyzed, and found to be consistent with the nucleotide sequence described in the reference, except for the primer region. The determined nucleotide sequence and its amino acid sequence are indicated by SEQ ID NO: 8 and SEQ ID NO: 16, respectively. The plasmid obtained was named pSE-BSA1 (FIG. 8).

### Example 15

### Enzyme Activity of the Transformant Transformed with a Plasmid Expressing Alcohol Dehydrogenase BstADHT Derived from Geobacillus stearothermophilus

A cell-free extract was prepared according to the above-described method using the Escherichia coli HB101 strain transformed with the pSE-BSA1 obtained in Example 1. The enzyme activity was measured according to the above-described method. The specific activity of BstADHT was 1.08 U/mg protein.

### Reference Example 1

### Cloning of Alcohol Dehydrogenase-I, ScADH1 Derived from Saccharomyces cerevisiae

S sense primer ScADH1-A1 (SEQ ID NO: 27) and an antisense primer ScADH1-T1 (SEQ ID NO: 28) were synthesized for cloning based on the nucleotide sequence (Accession No. M38456) of an alcohol dehydrogenase-I gene derived from Saccharomyces cerevisiae described in Basic Life Sci., 19, 335-361 (1982).

SEQ ID NO: 27

GTCGAATTCATACATGTCTATCCCAGAAACTCAAAAAGG

SEQ ID NO: 28

CTGCTTAAGTCTAGATTATTTAGAAGTGTCAACAACGTAACGACCAA

Using the chromosomal DNA obtained in Example 10, which had been purified from Saccharomyces cerevisiae, as the template, PCR was carried out in the same manner as in Example 4 using the primers ScADH1-A1 and ScADH1-T1.

The amplified product was double-digested with restriction enzymes EcoRI and AflII, and electrophoresed in an agarose gel. The band of interest was cut out, and then purified using GFX PCR DNA and Gel Band Purification Kit (Pharmacia).

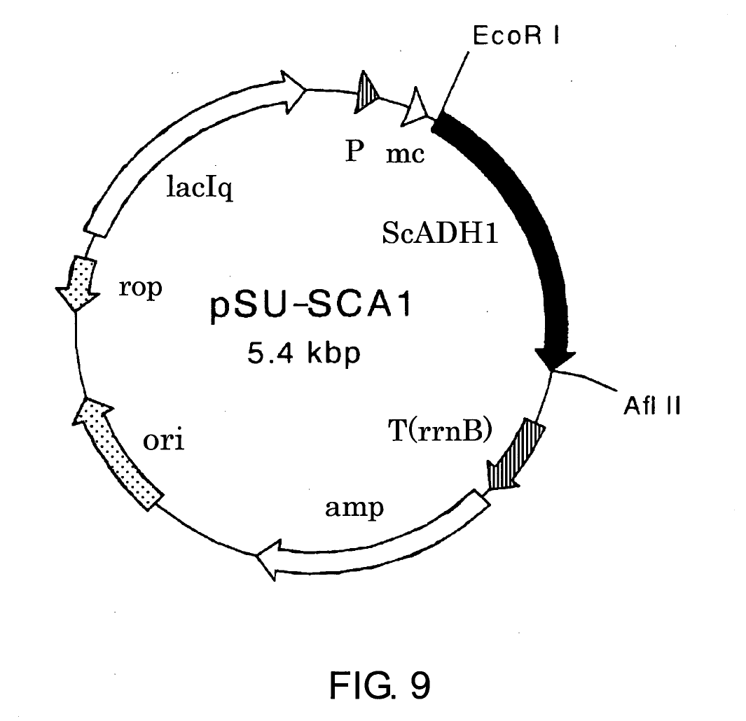

The resultant PCR-amplified DNA fragments after digestion with the restriction enzymes were ligated with the vector pSE420U (Japanese Patent Application No. 2005-169919), which had been double-digested with restriction enzymes EcoRI and AflII. Escherichia coli JM109 strain was transformed with the ligated DNA. The transformant was grown in an ampicillin-containing LB medium, and a plasmid was purified from the resultant transformant using FlexiPrep Kit (Pharmacia). The nucleotide sequence of the DNA insert of the purified plasmid was analyzed and compared with the nucleotide sequence described in the reference; except for the primer region, substitutions of ten bases and one amino acid were found. The determined nucleotide sequence and its amino acid sequence are indicated by SEQ ID NO: 29 and SEQ ID NO: 30, respectively. The plasmid obtained was named pSU-SCA1 (FIG. 9).

### Reference Example 2

### Enzyme Activity of the Transformant Transformed with a Plasmid Expressing Alcohol Dehydrogenase-I, ScADH1 Derived from Saccharomyces cerevisiae

A cell-free extract was prepared according to the above-described method using the Escherichia coli JM109 strain transformed with the pSU-SCA1 obtained in Reference Example 1. The enzyme activity was measured according to the above-described method. The specific activity of ScADH1 was 4.92 U/mg protein.

### Reference Example 3

### Cloning of Alcohol Dehydrogenase-II, ScADH2 Derived from Saccharomyces cerevisiae

A sense primer ScADH2-A1 (SEQ ID NO: 31) and an antisense primer ScADH2-T1 (SEQ ID NO: 32) were synthesized for cloning based on the nucleotide sequence (Accession No. J01314, M13475) of an alcohol dehydrogenase-II gene derived from Saccharomyces cerevisiae described in J. Biol. Chem., 258, 2674-2682 (1983).

SEQ ID NO: 31

GTCGAATTCATACATGTCTATTCCAGAAACTCAAAAAGC

SEQ ID NO: 32

GCACTTAAGTCTAGATTATTTAGAAGTGTCAACAACGTAACGACCAG

Using as the template the chromosomal DNA purified from Saccharomyces cerevisiae in Example 10, PCR was carried out in the same manner as in Example 4 using the primers ScADH2-A1 and ScADH2-T1.

The amplified product was double-digested with restriction enzymes EcoRI and AflII, and electrophoresed in an agarose gel. The band of interest was cut out, and then purified using GFX PCR DNA and Gel Band Purification Kit (Pharmacia).

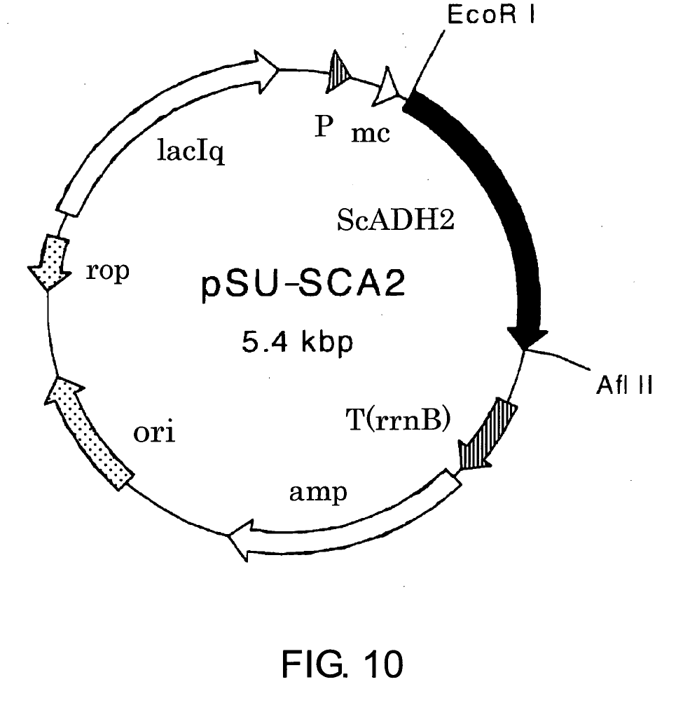

The resultant PCR-amplified DNA fragments after digestion with the restriction enzymes were ligated with the vector pSE420U (Japanese Patent Application No. 2005-169919), which had been double-digested with restriction enzymes EcoRI and AflII. Escherichia coli JM109 strain was transformed with the ligated DNA. The transformant was grown in an ampicillin-containing LB medium and a plasmid was purified from the resultant transformant using FlexiPrep Kit (Pharmacia). The nucleotide sequence of the DNA insert of the purified plasmid was analyzed and compared with the nucleotide sequence described in the reference; except for the primer region, substitution of one base was found. The determined nucleotide sequence and its amino acid sequence are indicated by SEQ ID NO: 33 and SEQ ID NO: 34, respectively. The plasmid obtained was named pSU-SCA2 (FIG. 10).

### Reference Example 4

### Enzyme Activity of the Transformant Transformed with a Plasmid Expressing Alcohol Dehydrogenase-II, ScADH2 Derived from Saccharomyces cerevisiae

A cell-free extract was prepared according to the above-described method using the Escherichia coli JM109 strain transformed with the pSU-SCA2 obtained in Reference Example 3. The enzyme activity was measured according to the above-described method. The specific activity of ScADH2 was 32.3 U/mg protein.

### Reference Example 5

### Cloning of Carbonyl Reductase ScGRE3 Derived from Saccharomyces cerevisiae

A sense primer GRE3-ATG1 (SEQ ID NO: 35) and an antisense primer GRE3-TAA1 (SEQ ID NO: 36) were synthesized for cloning, based on the nucleotide sequence (Accession No. U00059) of a carbonyl reductase gene derived from Saccharomyces cerevisiae described in Appl. Environ. Microbiol., 61, 1580-1585 (1995).

SEQ ID NO: 35

GAGTCATGAGTTCACTGGTTACTCTTAATAACGGTC

SEQ ID NO: 36

GACGAATTCCTCTAGATTATGCAAAAGTGGGGAATTTACCATC

Using the chromosomal DNA obtained in Example 10, which had been purified from Saccharomyces cerevisiae, as the template, PCR was carried out in the same manner as in Example 4 using the primers GRE3-ATG1 and GRE3-TAA1.

The amplified product was double-digested with restriction enzymes BspHI and EcoRI, and then electrophoresed in an agarose gel. The band of interest was cut out, and then purified using GFX PCR DNA and Gel Band Purification Kit (Pharmacia).

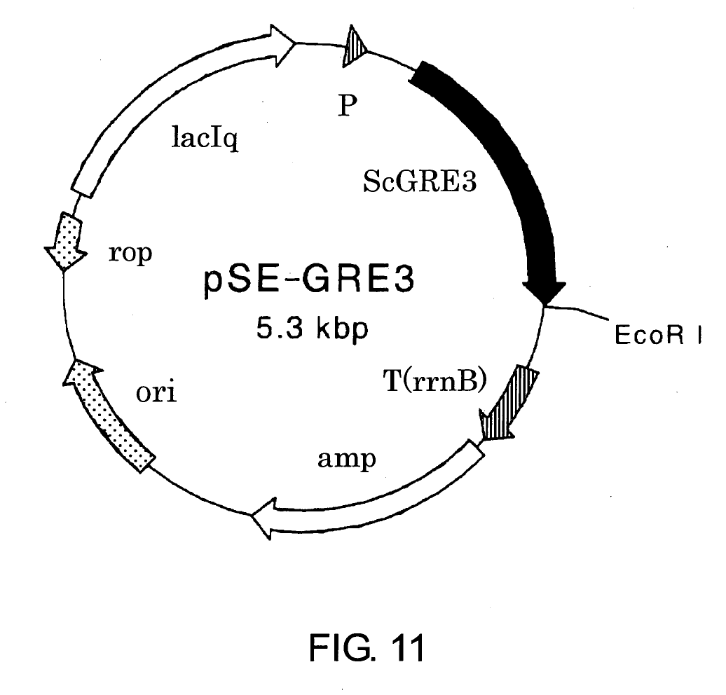

The resultant PCR-amplified DNA fragments after digestion with the restriction enzymes were ligated with the vector pSE420D (JP-A (Kokai) 2000-189170), which had been double-digested with restriction enzymes NcoI and EcoRI. Escherichia coli JM109 strain was transformed with the ligated DNA. The transformant was grown in an ampicillin-containing LB medium, and a plasmid was purified from the resultant transformant using FlexiPrep Kit (Pharmacia). The nucleotide sequence of the DNA insert of the purified plasmid was analyzed, and found to be consistent with the nucleotide sequence described in the reference. The determined nucleotide sequence and its amino acid sequence are indicated by SEQ ID NO: 37 and SEQ ID NO: 38, respectively. The plasmid obtained was named pSE-GRE3 (FIG. 11).

### Reference Example 6

### Enzyme Activity of the Transformant Transformed with a Plasmid Expressing Carbonyl Reductase ScGRE3 Derived from Saccharomyces cerevisiae

A cell-free extract was prepared according to the above-described method using the Escherichia coli JM109 strain transformed with the pSE-GRE3 obtained in Example 20. The enzyme activity was measured according to the above-described method. The specific activity of ScGRE3 was 206 mU/mg protein.

### Example 16

### Preparation of TFAC Hydrate

40 mL of 100 mM phosphate buffer solution (pH 7.4) was placed in 100-mL volumetric flask and cooled on an ice bath. To the flask 1,1,1-trifluoroacetone (TFAC) was added slowly and dissolved in the solution to make 100 mL, thereby obtaining a TFAC hydrate containing TFAC at a concentration of 944 g/L.

### Example 17

### Production of Optically Active TFIP Using TFAC as a Substrate

Escherichia coli HB101 strain was transformed with each of plasmids pSF-CPA4 (described in Example 1 herein), pSF-RED1 (described in Example 5 herein), pSF-SCP7 (described in Example 8 herein), pSF-ZRD1 (JP-A (Kokai) 2004-357639), and pSF-PFO2 (WO 01-061014), and the resultant transformants were cultured in a 2×YT medium (2% Bacto Tryptone, 1% Bacto Yeast Extract, 1% sodium chloride, and pH 7.2) overnight at 33° C. 0.1 mM IPTG was added to the medium to induce expression of each of the enzyme genes, and then the bacterial cells were collected to obtain Escherichia coli capable of expressing each of the enzyme genes.

The TFAC hydrate obtained in Example 16, 100 mM phosphate buffer solution (pH 6.5), two equivalents of sodium formate relative to TFAC, and bacterial cells collected from 10 g of the culture medium were placed in a reaction vessel to prepare a total of 10 g of reaction solution containing TFAC as the substrate at a final concentration of 2% (178 mM). The reaction solution was rotary shaken overnight at 20° C. 0.8 mL portions were sampled from the reaction solution, and extracted with 0.8 mL of dichloromethane. The organic layers were analyzed under the analysis conditions 1 and 2 described below to quantify TFIP and analyze its optical purity. The results are shown in Table 1.

TABLE 1

| Example, | | | Yield | Optical Purity | Absolute |
|---|---|---|---|---|---|
| Comparative Example | Plasmid | Enzyme | % | % e.e. | Configuration |
| Example 17 | pSF-CPA4 | CpSADH-McFDH | 67.8 | 99.1 | S |
| Example 17 | pSF-RED1 | ReSADH-McFDH | 63.0 | 98.7 | S |
| Example 17 | pSF-SCP7 | ScoPAR-McFDH | 49.4 | 98.5 | S |
| Example 17 | pSF-ZRD1 | ZraSBDH-McFDH | 64.3 | 97.8 | S |
| Example 18 | pSG-GCY1 | ScGCY1-BsGDH | 7.0 | 93.0 | S |
| Example 18 | pSG-HNR1 | HnTR1-BsGDH | 11.4 | 94.6 | S |
| Example 18 | pSG-DSR1 | DsTR1-BsGDH | 20.6 | 93.5 | S |
| Example 19 | pSE-BSA1 | BstADHT-Amano2 | 8.7 | 93.9 | S |
| Example 17 | pSF-PFO2 | PfODH-McFDH | 73.4 | 100.0 | R |
| Comparative Example 1 | pUC-SCA1 | ScADH1-Amano2 | 5.2 | 82.3 | S |
| Comparative Example 1 | pUC-SCA2 | ScADH2-Amano2 | 8.9 | 58.0 | S |
| Comparative Example 1 | pSE-GRE3 | ScGRE3-Amano2 | 8.3 | 69.6 | S |

### Example 18

### Production of Optically Active TFIP Using TFAC as a Substrate

Escherichia coli HB101 strain was transformed with each of the plasmids pSG-GCY1 (described in Example 12 herein), pSG-HNR1 (JP-A (Kokai) 2003-230398), and pSG-DSR1 (JP-A (Kokai) 2003-230398), and the resultant transformants were cultured in a 2×YT medium (2% Bacto Tryptone, 1% Bacto Yeast Extract, 1% sodium chloride, and pH 7.2) overnight at 33° C. 0.1 mM IPTG was added to the medium to induce expression of each of the enzyme genes, and then the bacterial cells were collected to obtain Escherichia coli expressing each of the enzyme genes.

The TFAC hydrate obtained in Example 16, 100 mM phosphate buffer solution (pH 6.5), two equivalents of D-glucose relative to TFAC, and bacterial cells collected from 10 g of the culture medium were placed in a reaction vessel to prepare a total of 10 g of reaction solution containing TFAC as the substrate at a final concentration of 2% (178 mM). The reaction solution was rotary shaken overnight at 20° C. 0.8 mL portions were sampled from the reaction solution, and extracted with 0.8 mL of dichloromethane. The organic layers were analyzed under the analysis conditions 1 and 2 described below to quantify TFIP and analyze its optical purity. The results are shown in Table 1.

### Example 19

### Production of Optically Active TFIP Using TFAC as a Substrate

Escherichia coli HB101 strain was transformed with a plasmid pSE-BSA1 (described in Example 14 herein), and the resultant transformant was cultured in a 2×YT medium (2% Bacto Tryptone, 1% Bacto Yeast Extract, 1% sodium chloride, and pH 7.2) overnight at 33° C. 0.1 mM IPTG was added to the medium to induce expression of each of the enzyme genes, and then the bacterial cells were collected to obtain Escherichia coli expressing each of the enzyme genes.

The TFAC hydrate obtained in Example 16, 100 mM phosphate buffer solution (pH 6.5), two equivalents of D-glucose relative to TFAC, bacterial cells collected from 10 g of the culture medium, and 5 U glucose dehydrogenase (Amano-2, Amano Enzyme Inc.) were placed in a reaction vessel to prepare a total of 10 g of reaction solution containing TFAC as the substrate at a final concentration of 2% (178 mM). The reaction solution was rotary shaken overnight at 20° C. 0.8 mL portions were sampled from the reaction solution, and extracted with 0.8 mL of dichloromethane. The organic layers were analyzed under the analysis conditions 1 and 2 described below to quantify TFIP and analyze its optical purity. The results are shown in Table 1.

### Example 20

### Production of (S)-TFIP Using TFAC as a Substrate

Escherichia coli HB101 strain was transformed with a plasmid pSF-CPA4 (described in Example 1 herein), and the resultant transformant was cultured in a 2×YT medium (2% Bacto Tryptone, 1% Bacto Yeast Extract, 1% sodium chloride, and pH 7.2) overnight at 33° C. 0.1 mM IPTG was added to the medium to induce expression of each of the enzyme genes, and then the bacterial cells were collected to obtain Escherichia coli coexpressing CpSADH and McFDH.

The TFAC hydrate obtained in Example 20, 100 mM phosphate buffer solution (pH 6.0), 803 mM sodium formate, and bacterial cells collected from 267 g of the culture medium were placed in a reaction vessel to prepare a total of 400 g of reaction solution containing TFAC as the substrate at a final concentration of 6% (535 mM). The reaction solution was allowed to react at 20° C. under stirring with the pH controlled at 6.0 with 40% sulfuric acid. After a lapse of 26 hours, 0.8 mL portions were sampled from the reaction solution, and extracted with 0.8 mL of dichloromethane. The organic layers were analyzed under the analysis conditions 1 and 2 described below to quantify TFIP and analyze its optical purity. The conversion rate was 99% and optical purity was 99.8% e.e. (S).

### Example 21

### Purification of (S)-TFIP

Further, 1230 g of the reaction solution obtained in Example 20 was centrifuged thereby removing bacterial cells. The resultant supernatant was distilled, and the distillate at a vapor temperature of 77-79° C. was collected as the main fraction in a yield of 60.17 g. To 59.02 g of the main fraction, 11.8 mL of a saturated calcium chloride aqueous solution was added, and the mixture was stirred for one hour at room temperature. Thereafter, the mixture was allowed to stand to separate the organic layer. The organic layer was distilled again, and the distillate at a vapor temperature of 72-74° C. was collected as the main fraction in a yield of 48.63 g. The main fraction was analyzed under the analysis conditions 1 and 2; the TFIP purity was 97.3% and optical purity was 99.7% e.e. (S).

### Comparative Example 1

### Production of (S)-TFIP Using TFAC as a Substrate

Escherichia coli HB01 strain was transformed with plasmids pSU-SCA1 (described in Reference Example 1 herein), pSU-SCA2 (described in Reference Example 3 herein), and pSE-GRE3 (described in Reference Example 5 herein), and the resultant transformants were cultured in a 2×YT medium (2% Bacto Tryptone, 1% Bacto Yeast Extract, 1% sodium chloride, and pH 7.2) overnight at 33° C. 0.1 mM IPTG was added to the medium to induce expression of each of the enzyme genes, and then the bacterial cells were collected to obtain Escherichia coli expressing each of the enzyme genes.

The TFAC hydrate obtained in Example 20, 100 mM phosphate buffer solution (pH 6.5), two equivalents of D-glucose relative to TFAC, bacterial cells collected from 10 g of the culture medium, and 5 U glucose dehydrogenase (Amano-2, Amano Enzyme Inc.) were placed in a reaction vessel to prepare a total of 10 g of reaction solution containing TFAC as the substrate at a final concentration of 2% (178 mM). The reaction solution was rotary shaken overnight at 20° C. 0.8 mL portions were sampled from the reaction mixture, and extracted with 0.8 mL of dichloromethane. The organic layers were analyzed under the analysis conditions 1 and 2 described below to quantify TFIP and analyze its optical purity. The results are shown in Table 1.

### Comparative Example 2

### Influence of Reaction pH on Optical Purity

Reaction was carried out in the same manner as in Example 20, except that the final concentration of the substrate added to the reaction solution was 5% (446 mM), the pH of the phosphate buffer solutions was 6.5, and the pH during the reaction was controlled at 6.5. The resultant optical purity was 99.1-99.2% e.e. (S).

### INDUSTRIAL APPLICABILITY

The present invention provides methods for enzymatically producing optically active (S)-1,1,1-trifluoro-2-propanol and (R)-1,1,1-trifluoro-2-propanol. Both (S)-1,1,1-trifluoro-2-propanol and (R)-1,1,1-trifluoro-2-propanol produced by the method of the present invention have high optical purity. That is, compared to a conventional method that uses an optical resolution agent, higher optical purity can be accomplished more easily and at lower cost.

Furthermore, according to the present invention, the compound of interest can be obtained efficiently using a large amount of raw material. More specifically, the present invention enables, for example, convenient and economical industrial production of (S)-1,1,1-trifluoro-2-propanol and (R)-1,1,1-trifluoro-2-propanol. That is, substances of the present invention with enzymatic activities are not easily inhibited by large amount of substrate and also by the reaction products produced from the substrate. Therefore, the present invention enables use of a larger amount of raw material to efficiently obtain the compound of interest.

(S)-1,1,1-trifluoro-2-propanol and (R)-1,1,1-trifluoro-2-propanol produced by the method of the present invention will be useful as optically active raw materials for various types of pharmaceutical products, liquid crystalline materials, and such.

1. A method for producing (S)-1,1,1-trifluoro-2-propanol represented by formula (2), 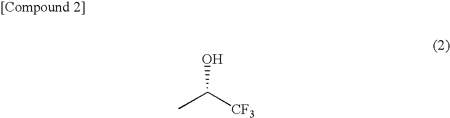
which comprises the step of reacting a protein encoded by the polynucleotide of any one of the following (a) to (e), a microorganism or a transformant strain that functionally expresses said protein, or a processed material thereof, with 1,1,1-trifluoroacetone represented by formula (1): 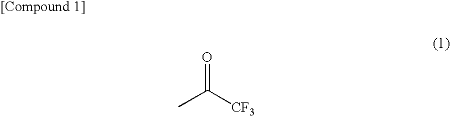
(a) a polynucleotide comprising the nucleotide sequence of any one of SEQ ID NOs: 1 to 8;
(b) a polynucleotide encoding a protein comprising the amino acid sequence of any one of SEQ ID NOs: 9 to 16;
(c) a polynucleotide encoding a protein comprising amino acids with one or more amino acid substitutions, deletions, insertions, and/or additions in the amino acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2);
(d) a polynucleotide that hybridizes under stringent conditions with a DNA comprising the nucleotide sequence of any one of SEQ ID NOs: 1 to 8, wherein the polynucleotide encodes a protein having an activity of reducing 1,1,1-trifluoroacetone represented by formula (1) to produce (S)-1,1,1-trifluoro-2-propanol represented by formula (2); and
(e) a polynucleotide encoding a protein comprising an amino acid sequence having 80% or more homology to the amino acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2).

2. A method for producing (R)-1,1,1-trifluoro-2-propanol represented by formula (3), 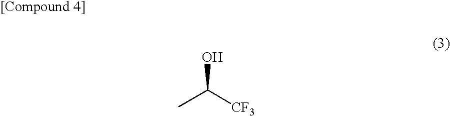
which comprises the step of reacting a protein encoded by the polynucleotide of any one of the following (a) to (e), a microorganism or a transformant strain that functionally expresses said protein, or a processed material thereof, with 1,1,1-trifluoroacetone represented by formula (1): 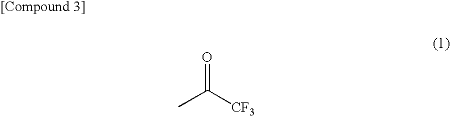
(a) a polynucleotide comprising the nucleotide sequence of SEQ ID NO: 17;
(b) a polynucleotide encoding a protein comprising the amino acid sequence of SEQ ID NO: 18;
(c) a polynucleotide encoding a protein comprising amino acids with one or more amino acid substitutions, deletions, insertions, and/or additions in the amino acid sequence of SEQ ID NO: 18, wherein the protein has an activity of reducing 1,1,1-trifluoroacetone represented by formula (1) to produce (R)-1,1,1-trifluoro-2-propanol represented by formula (3);
(d) a polynucleotide that hybridizes under stringent conditions with a DNA comprising the nucleotide sequence of SEQ ID NO: 17, wherein the polynucleotide encodes a protein having an activity of reducing 1,1,1-trifluoroacetone represented by formula (1) to produce (R)-1,1,1-trifluoro-2-propanol represented by formula (3); and
(e) a polynucleotide encoding a protein comprising an amino acid sequence having 80% or more homology to the amino acid sequence of SEQ ID NO: 18, wherein the protein has an activity of reducing 1,1,1-trifluoroacetone represented by formula (1) to produce (R)-1,1,1-trifluoro-2-propanol represented by formula (3).

3. A method for producing (S)-1,1,1-trifluoro-2-propanol represented by formula (2), 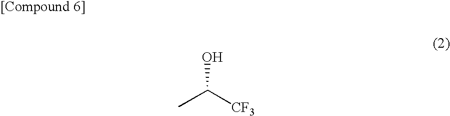
which comprises the step of reacting a protein encoded by the polynucleotide of any one of the following (a) to (e), a transformant strain that coexpresses a coenzyme corresponding to said protein and a dehydrogenase having an activity to regenerate reduced nicotinamide adenine dinucleotide (NADH) or reduced nicotinamide adenine dinucleotide phosphate (NADPH), or a processed material thereof, with 1,1,1-trifluoroacetone represented by formula (1): 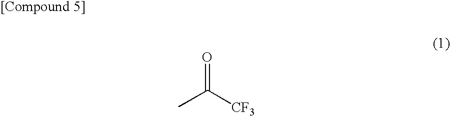
(a) a polynucleotide comprising the nucleotide sequence of any one of SEQ ID NOs: 1 to 8;
(b) a polynucleotide encoding a protein comprising the amino acid sequence of any one of SEQ ID NOs: 9 to 16;
(c) a polynucleotide encoding a protein comprising amino acids with one or more amino acid substitutions, deletions, insertions, and/or additions in the amino acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2);
(d) a polynucleotide that hybridizes under stringent conditions with a DNA comprising the nucleotide sequence of any one of SEQ ID NOs: 1 to 8, wherein the polynucleotide encodes a protein having an activity of reducing 1,1,1-trifluoroacetone represented by formula (1) to produce (S)-1,1,1-trifluoro-2-propanol represented by formula (2); and
(e) a polynucleotide encoding a protein comprising an amino acid sequence having 80% or more homology to the amino acid sequence of any one of SEQ ID NOs: 9 to 16, wherein the protein has an activity of reducing 1,1,1-trifluoroacetone represented by formula (1) to produce (S)-1,1,1-trifluoro-2-propanol represented by formula (2).

4. A method for producing (R)-1,1,1-trifluoro-2-propanol represented by formula (3), 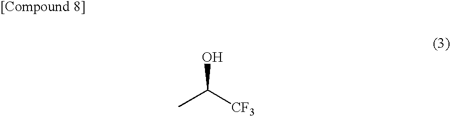
which comprises the step of reacting a protein encoded by the polynucleotide of any one of the following (a) to (e), a transformant strain that coexpresses a coenzyme corresponding to said protein and a dehydrogenase having an activity to regenerate reduced nicotinamide adenine dinucleotide (NADH) or reduced nicotinamide adenine dinucleotide phosphate (NADPH), or a processed material thereof, with 1,1,1-trifluoroacetone represented by formula (1): 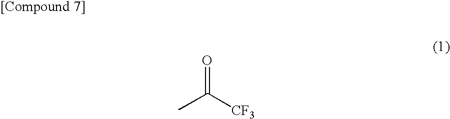
(a) a polynucleotide comprising the nucleotide sequence of SEQ ID NO: 17;
(b) a polynucleotide encoding a protein comprising the amino acid sequence of SEQ ID NO: 18;
(c) a polynucleotide encoding a protein comprising amino acids with one or more amino acid substitutions, deletions, insertions, and/or additions in the amino acid sequence of SEQ ID NO: 18, wherein the protein has an activity of reducing 1,1,1-trifluoroacetone represented by formula (1) to produce (R)-1,1,1-trifluoro-2-propanol represented by formula (3);
(d) a polynucleotide that hybridizes under stringent conditions with a DNA comprising the nucleotide sequence of SEQ ID NO: 17, wherein the polynucleotide encodes a protein having an activity of reducing 1,1,1-trifluoroacetone represented by formula (1) to produce (R)-1,1,1-trifluoro-2-propanol represented by formula (3); and
(e) a polynucleotide encoding a protein comprising an amino acid sequence having 80% or more homology to the amino acid sequence of SEQ ID NO: 18, wherein the protein has an activity of reducing 1,1,1-trifluoroacetone represented by formula (1) to produce (R)-1,1,1-trifluoro-2-propanol represented by formula (3).

5. The method of claim 3 or 4, wherein the dehydrogenase having an activity to regenerate reduced nicotinamide adenine dinucleotide (NADH) or reduced nicotinamide adenine dinucleotide phosphate (NADPH) is glucose dehydrogenase or formate dehydrogenase.

6. A method for stably producing (S)-1,1,1-trifluoro-2-propanol represented by formula (2) 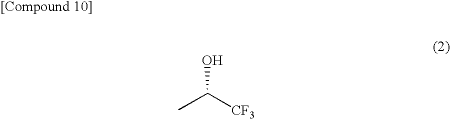
with an optical purity of 99.5% e.e. or more by reacting a protein encoded by the polynucleotide of any one of the following (a) to (e), a microorganism or a transformant strain that functionally expresses said protein, or a processed material thereof, with 1,1,1-trifluoroacetone represented by formula (1) within a pH range of 5.0 to 6.4: 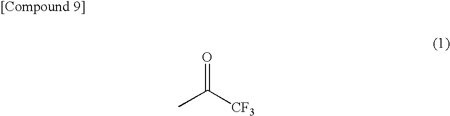
(a) a polynucleotide comprising the nucleotide sequence of SEQ ID NO: 1;
(b) a polynucleotide encoding a protein comprising the amino acid sequence of SEQ ID NO: 9;
(c) a polynucleotide encoding a protein comprising amino acids with one or more amino acid substitutions, deletions, insertions, and/or additions in the amino acid sequence of SEQ ID NO: 9, wherein the protein has an activity of reducing 1,1,1-trifluoroacetone represented by formula (1) to produce (S)-1,1,1-trifluoro-2-propanol represented by formula (2);
(d) a polynucleotide that hybridizes under stringent conditions with a DNA comprising the nucleotide sequence of SEQ ID NO: 1, wherein the polynucleotide encodes a protein having an activity of reducing 1,1,1-trifluoroacetone represented by formula (1) to produce (S)-1,1,1-trifluoro-2-propanol represented by formula (2); and
(e) a polynucleotide encoding a protein comprising an amino acid sequence having 80% or more homology to the amino acid sequence of SEQ ID NO: 9, wherein the protein has an activity of reducing 1,1,1-trifluoroacetone represented by formula (1) to produce (S)-1,1,1-trifluoro-2-propanol represented by formula (2).
